(19) United States
(12) Patent Application Publication
(10) Pub. No.: US 2005/0197545 A1
(43) Pub. Date: Sep. 8, 2005

(54) System and method for disease management

(76) Inventor: John M. Hoggle, Douglasville, GA (US)

Correspondence Address: MYERS & KAPLAN, INTELLECTUAL PROPERTY LAW, L.L.C., 1899 POWERS FERRY ROAD, SUITE 310, ATLANTA, GA, 30339, US

(21) Appl. No.: 11/069,257

(22) Filed: Mar. 1, 2005

Related U.S. Application Data
(60) Provisional application No. 60/549,209, filed on Mar. 2, 2004.

(51) Int. Cl. A61B 5/00; A61B 10/00; G06F 17/00; G06K 9/62

(52) U.S. Cl. 600/300; 128/920; 128/923

(57) ABSTRACT

A system and method for disease management that effectively eliminates unnecessary layers and/or intermediaries within the healthcare management system by enabling a disease management entity to operate as an extension of a physician's office, and thus, work closely with the patients and the patient's physicians to effectively empower patients towards self-care and a higher level of awareness and understanding of their disease process and treatment. The present system and method utilizes a team of highly trained healthcare professionals and technicians working together with patients and physicians, wherein remote vital sign monitoring equipment, software systems, and standing orders and treatment protocols, are utilized to effectively manage patients with chronic conditions. Additionally, as the disease management entity operates as an extension of a physician's office, the disease management entity is able to coordinate with a payor through the physician's office, and thus provide the payor with positive financial outcomes.

### CROSS-REFERENCE AND PRIORITY CLAIM TO RELATED APPLICATIONS

To the fullest extent permitted by law, the present nonprovisional patent application claims priority to and the full benefit of provisional patent application entitled “SYSTEM AND METHOD FOR DISEASE MANAGEMENT”, filed on Mar. 2, 2004, having assigned Ser. No. 60/549,209.

### COPYRIGHT NOTICE

A portion of the disclosure of this patent document contains material which is subject to copyright protection. The copyright owner has no objection to the facsimile reproduction by anyone of the patent document or patent disclosure as it appears in the U.S. Patent and Trademark Office patent file or records, but otherwise reserves all copyright rights whatsoever.

### TECHNICAL FIELD

The present invention relates generally to methods of disease management and patient care, and more specifically to a system and method for disease management, wherein the present invention is particularly advantageous for its ability to eliminate extraneous layers and/or intermediaries commonly associated with conventional healthcare delivery systems, and thus, reduce unnecessary healthcare costs, avoid potential misallocation of healthcare resources, improve clinical outcomes for the patient, and increase physician efficiency in the delivery of patient healthcare services.

### BACKGROUND OF THE INVENTION

Heart failure occurs when the heart loses its ability to effectively pump a sufficient quantity of blood through the body. Usually, the loss in pumping action is a symptom of an underlying heart problem, such as coronary artery disease, abnormal or faulty heart valves, cardiomyopathy, severe lung disease, diabetes, anemia, or the like.

Although the term heart failure suggests a sudden and complete stop of heart activity, the heart does not actually suddenly stop. Instead, heart failure usually develops slowly, often over years, as the heart gradually loses its pumping ability and, as result, functions less efficiently. Some individuals may not become aware of their heart condition until symptoms appear—typically years after their heart began its decline.

The severity of a heart condition is in large part dependent upon how much pumping capacity the heart has actually lost. Although nearly all individuals will lose some pumping capacity as they progress in age, the loss of pumping capacity is significantly more in heart failure patients. Naturally, the severity of the condition further determines the impact it may have on a particular individual's life. At one end of the spectrum, a mild form of heart failure may have little effect on the individual's life; at the other end, however, severe heart failure can interfere with even the simplest tasks and/or daily activities, and even prove fatal.

Accordingly, in an effort to promote a healthier lifestyle and prevent the exacerbation of existing heart conditions, patients are encouraged to seek regular professional treatment and/or enroll in a disease management program adapted to educate the patient on the details of his/her disease, and ultimately lead to a patient-implemented self-management process.

Unfortunately however, although a variety of disease management programs and/or approaches attempt to manage a variety of chronic diseases, including heart disease, diabetes and asthma, such available disease management programs share inherent disadvantages that render implementation of same largely ineffective, impractical, overly expensive, and inefficient.

Specifically, the traditional model of disease management positions itself as a separate layer of medical service outside of the normal patient-physician-payor model of healthcare delivery. Beginning with the payor's medical database, patients are identified and contacted directly by the disease management entity, either by a department within the payor's operations or an independent disease management company, and thereafter, enroll the patient within an associated disease management program. Disadvantageously however, by contacting the patients directly, the disease management company leaves the patient's physician out of the process, and, as such, do not provide a process for physician or clinical intervention with the patient. The traditional model instead primarily focuses on patient education, and attempts to alter patient behavior in hopes of leading to a better understanding of the disease process and early recognition of exacerbation. However, under such traditional models, the only mechanism for treating patients who remain clinically unresponsive and/or non-compliant is to refer them to their physician or healthcare institution during times of crisis, which increases overall healthcare costs for the patient, healthcare provider, and payor, as the physician must expend significant time and resources familiarizing him/herself with the patient's medical history.

Moreover, the structures of traditional disease management processes vary greatly. Some disease management processes rely only on education, whereas others may rely on patient self-monitoring and/or overly expensive home-based care and visitations. Notably, it is precisely this variation in the structure of disease management processes that has resulted in disease management studies reporting a substantial variation in results. Unfortunately, the majority of such results typically illustrate seemingly avoidable high healthcare costs, an increase in cost of direct patient care services, a reduction in effective patient clinical outcomes, and placement of significant resource burdens on the healthcare system by consuming physician management time, nursing hours, hospital beds and other financial assets of the healthcare system.

Therefore, it is readily apparent that there is a need for a system and method for disease management, wherein the present invention eliminates extraneous layers and/or intermediaries commonly associated with conventional disease management processes or healthcare delivery systems in general, and, instead, effectively improves patient clinical outcomes, while providing the payor with positive financial outcomes, and significantly reducing, or eliminating, the misallocation of healthcare resources by enabling physicians to effectively manage time and provide patient healthcare services in a cost-efficient and effective manner.

### BRIEF SUMMARY OF THE INVENTION

Briefly described, in a preferred embodiment, the present invention overcomes the above-mentioned disadvantages and meets the recognized need for such a method by providing a system and method for disease management that effectively eliminates unnecessary layers and/or intermediaries within the healthcare management system by enabling a disease management entity to operate as an extension of a physician's office, and thus, work closely with the patients and the patient's physicians to effectively empower patients toward self-care and a higher level of awareness and understanding of their disease process and treatment. The present system and method utilizes a team of highly trained healthcare professionals and technicians working together with patients and physicians, wherein state-of-the-art telemonitoring equipment, remote vital sign monitors, proprietary custom-built software systems, and standing orders and treatment protocols that meet published best practice guidelines, are utilized to effectively manage patients with chronic conditions; thus, providing each patient with a comprehensive care program that will promote preventative wellness, improve clinical outcomes, reduce unnecessary healthcare costs and enhance quality of life for patients, families and caregivers. All patients are recruited into the disease management program through their physicians, wherein patients may discuss disease management program options in private doctor-patient conversations where confidentiality is assured. Additionally, as the disease management entity operates as an extension of a physician's office, the disease management entity is able to coordinate with a payor through the physician's office, and thus provide the payor with positive financial outcomes.

According to its major aspects and broadly stated, the present invention in its preferred form is a system and method for disease management incorporating a software management program implemented via a local network and/or global networking system for managing all aspects of the present system and method including clinical, billing, inventory, and clinical outcomes and financial reporting, and wherein the system and method further preferably generally includes a network of healthcare professionals, remote vital sign monitors for receiving and transmitting objective and subjective physiological data from the patient, and standing orders and/or treatment protocols for systematic implementation of same based upon the patient's physiological data transmitted by the remote vital signs monitors to the network of healthcare professionals.

More specifically, the present invention is a system and method for disease management that provides a clinical information management system for utilization by patients, payors, physicians, and ancillary health care providers, to collect, communicate and analyze patient specific data, and implement physician-directed medical interventions for the resolution, treatment and management of patient disease conditions.

Preferably, simple to use FDA-approved electronic monitoring and collection devices are placed within a patient's home for facilitating the automated transmission, over standard phone lines or the like, of daily objective and subjective patient physiological data between the patient and a team of medical professionals (i.e., critical care cardiac nurses, technicians, and the like) of a disease management entity working in association with the patient's physician(s). The patient preferably transmits daily physiological data, including blood pressure, pulse, and weight, to an electronic data warehouse accessible by the team of medical professionals of the disease management entity, the patient's physician, and the patient him/herself.

Preferably, best practice guidelines, developed by medical consultants and based on guidelines of the American College of Cardiology and American Heart Association, are utilized by the patient's physician to develop standing orders and treatment protocols that are entered into the system and data warehouse for each patient. Based upon such standing orders and protocols, and the information in the data warehouse, nurses from the medical professional team respond with clinical intervention for patients whose data falls outside set clinical parameters (i.e., unscheduled interventions). For example, if the patient's weight increases beyond a certain threshold, the nurse may, within limits set by the standing orders, increase diuretic medications. In the event the patient's physiological data remains outside normal parameters, a nurse may directly consult with the patient's attending physician. If the patient requires hospitalization for worsening symptoms, the nurse coordinates with the patient's physician and hospital to facilitate a direct admission for the patient, thereby avoiding costly admissions to the emergency department prior to hospitalization. Even when measurements or data remain normal, the nurse preferably contacts the patient to conduct routine assessments (i.e., scheduled interventions). Preferably, the patient's disease state determines the frequency of the routine or scheduled assessments/interventions—typically conducted on each patient between one and three times per week.

Preferably, during each contact with the patient, scheduled or unscheduled, a nurse assessment is made, wherein the assessment is scripted, and the results entered into the data warehouse for future access (via a secure global networking connection or other web access means) by the patient, the patient's physician, the payor, and/or disease management entity. Patients further have access to the nurses of the disease management medical professional team twenty-four hours a day, seven days per week. Notably, it is this clinical intervention process of the present system and method that yields significantly more effective results over traditional disease management models.

The present system and method contemplates application to those patients with poor ventricular function (i.e., systolic dysfunction), diastolic dysfunction, multiple co-morbidities, cardiac transplants, idiopathic and ischemic cardiomyopathy, and patients with varying levels of acuity (i.e., New York Heart Association classification of I, II, III and/or IV). Additionally, the present invention provides various disease management levels, including complex management, intermediate management, basic management, and maintenance, wherein each management level/program preferably seeks to reduced patient hospitalizations, prolong patient life, increase functional capacity, stabilize and slow progression of patient disease, reduce diagnostic testing, and decrease progression into myocardial infarction and coronary artery bypass graphting.

The present system and method further preferably incorporates patient education. Specifically, with each patient contact, the nurses of the disease management medical team will teach each patient about good health practices, nutrition, and heart disease. For example, many patients do not understand the role salt plays in their diet. As such, the present system and method contemplates the provision of written informational packages for distribution to patients, thereby educating the patients on healthy practices. Furthermore, since the nurses are frequently in contact with the patients (i.e., via scheduled and/or unscheduled interventions and contacts), the present system and method provides an ongoing and customized personal education campaign for each patient.

Preferably, the present system and method further provides a complete suite of proprietary software applications for managing all aspects of system operations, including clinical, billing, inventory, and clinical outcomes and financial reporting. Specifically, a patient care management software system and application preferably provides a variety of documentation for the patient's individual treating physician medical care plan, the objective and assessment data collected, the clinical interventions provided to the patient based on the physician's standing medical orders, the results of medical interventions provided to the patient, the communication of clinical information to the patients, their physician, and their payor, and the clinical status of the patients through scoring of clinical parameters. The patient care management software system and application of the present invention not only serves as the central data collection point for patient management and data collection, but is also utilized to track and report outcomes to payors, physicians, and patients.

Additionally, the present invention further provides a web-based patient care management software system and application that comprises the majority of features available on the patient care management software system and application described above, but is conveniently accessible through a global networking system (i.e., World Wide Web, or the like). User access to the web-based application is preferably restricted to the disease management medical professional team, member physicians and enrolled patients, with each group of users accessing a distinct interface. Moreover, access to the web-based application is preferably monitored, logged and audited as an additional layer of security. The system further preferably provides multiple reporting options, including web-based access for patients and physicians, and periodic reports in either hardcopy and/or electronic-mail format delivered to the patient's physicians.

Implementation of the present system and method further preferably seeks to improve the efficiency and quality of physician-delivered healthcare, and thus significantly improve the financial and clinical outcomes for “at risk” healthcare providers (i.e., hospitals), “at risk” third party payors (i.e., Medicare HMOs, HMOs, PPOs, and the like), and “at risk” government healthcare providers/payors (VA system, CHAMPUS, and the like). Specifically, the vast majority of hospitals suffer significant financial loss on patients admitted into the hospital system with a diagnosis of congestive heart failure, as such patient admissions are characterized by frequent and lengthy stays that exceed the reimbursement rates of the hospital. Such patients present additional risk to a hospital provider, as such patients are frequently readmitted before the reimbursement guidelines take effect, sending the hospital provider into a non-reimbursement status for each such patient. Further, most such patients frequently consume resources beyond simple financial considerations, such as physician and medical staff time. Unfortunately, a hospital's financial impact for admission of such congestive heart failure patients results in significant cost to third party payors. Specifically, the perception of patients who are treated within the traditional managed care systems is a feeling of isolation from the healthcare system and a perceived risk of receiving sub-standard care from the entire healthcare system. The perception of physicians within the managed healthcare system is that they are seeing more patients, receiving less reimbursement, and having less time to make critical medical decisions—potentially cumulatively lending to frequent readmission of the patients back into the hospital system; thus resulting in the above-discussed financial impact to hospital providers.

As such, the present system and method functions to provide a direct connection between the treating physician and the patient for the collection of relevant patient data for effectively satiating medical and subjective needs. That is, the present system embraces the treating physician—meeting the physician on his/her own turf by providing efficient and expeditious patient data, and answers to complex time-consuming questions. Moreover, the present system enables the patient and physician to be in constant contact via the continuous and remote monitoring and transmission of patient critical physiological functions and changes in subjective symptoms (i.e., clinical information), wherein the present system may be utilized to consolidate and package such clinical information with the intent of providing the physician with immediate need-to-know documentation based upon a physician-designed patient healthcare management plan. As a result of the present system, patients will be connected to the system both in perception and in reality; the patient will receive timely medical interventions that will avert physically and financially costly procedures and admissions; and, the patient's individual treating physician(s) will be connected to the system, thereby allowing a more efficient treatment plan to be implemented. Furthermore, the treating physicians will also benefit by providing for expanded reimbursement opportunities for their practice, as well as provide a platform for greater flexibility in treating more patients more efficiently. Moreover, payors will be provided with a system for creating an efficient operation that cooperates with local treating physicians and, at the same time, enables payor participation in a system that provides the healthcare system with significantly better financial and clinical outcomes.

Accordingly, a feature and advantage of the present invention is its ability to enable a disease management entity to operate as an extension of a physician's office in the treatment and management of patients suffering from chronic diseases.

Another feature and advantage of the present invention is its direct physician involvement throughout the disease management system and program, including initial enrollment of patients into the program, in view of traditional disease management programs that in large part, or completely, bypass or exclude physician participation and involvement, thus increasing overall healthcare inefficiencies and financial loss.

Still another feature and advantage of the present invention is its daily, automated collection and analysis of objective patient physiological data.

Still yet another feature and advantage of the present invention is its utilization of standing orders and protocols based on best practices guidelines for effective and efficient patient care management and clinical interventions.

A further feature and advantage of the present invention is its ability to improve quality of life for the patient by enabling the development of extensive clinical data for the physician-directed formulation of efficient methods for tracking and treating the patient.

Still a further feature and advantage of the present invention is its ability to enable a disease management entity to operate as an extension of a physician's office, thereby enabling the disease management entity to coordinate with a payor through the physician's office, and thus provide the payor with positive financial outcomes.

Still yet a further feature and advantage of the present invention is its ability to provide an ongoing and customized personal education campaign for each patient.

Still another and further feature and advantage of the present invention is its ability to provide a complete suite of proprietary software applications for managing all aspects of system operations, including clinical, billing, inventory, and clinical outcomes and financial reporting.

Yet another and further feature and advantage of the present invention is its ability to provide a web-based patient care management software system and application conveniently accessible through a global networking system.

Still yet another and further feature and advantage of the present invention is its ability to improve the efficiency and quality of physician-delivered healthcare, and thus significantly improve the financial and clinical outcomes for “at risk” healthcare providers, “at risk” third party payors, and “at risk” government healthcare providers/payors.

Still another feature and advantage of the present invention is its provisions of a network of healthcare professionals, remote vital sign monitors for receiving and transmitting objective and subjective physiological data from the patient, and standing orders and/or treatment protocols for systematic implementation of same based upon the patient's transmitted physiological data and physician-directed disease management plans.

Yet another feature and advantage of the present invention is its ability to reduce in-hospital admissions, reduce in-hospital emergency room and/or clinic visits related to congestive heart failure, reduce in-home health visits related to congestive heart failure, and reduce unscheduled physician visits related to congestive heart failure.

These and other features and advantages of the present invention will become more apparent to one skilled in the art from the following description and claims when read in light of the accompanying drawings.

### BRIEF DESCRIPTION OF THE DRAWINGS

The present invention will be better understood by reading the Detailed Description of the Preferred and Alternate Embodiments with reference to the accompanying drawing figures, in which like reference numerals denote similar structure and refer to like elements throughout, and in which:

FIG. 1 is a flow diagram illustrating a method for patient enrollment into the present system and method for disease management according to a preferred embodiment of the present invention;

FIG. 2 is a flow diagram illustrating a method for patient treatment in the present system and method for disease management according to a preferred embodiment of the present invention;

FIG. 3 illustrates an exemplary embodiment of a patient log sheet for utilization within the present system and method for disease management according to a preferred embodiment of the present invention;

FIG. 4 illustrates an exemplary embodiment of a disease management protocol and standing order guideline for utilization within the present system and method for disease management according to a preferred embodiment of the present invention;

FIG. 5 illustrates an exemplary embodiment of a disease management protocol and standing order guideline for utilization within the present system and method for disease management according to a preferred embodiment of the present invention;

FIG. 6 illustrates an exemplary embodiment of a disease management protocol and standing order guideline for utilization within the present system and method for disease management according to a preferred embodiment of the present invention;

FIG. 7 illustrates an exemplary embodiment of a disease management protocol and standing order guideline for utilization within the present system and method for disease management according to a preferred embodiment of the present invention;

FIG. 8 illustrates an exemplary embodiment of a disease management protocol and standing order guideline for utilization within the present system and method for disease management according to a preferred embodiment of the present invention;

FIG. 9 illustrates an exemplary embodiment of a disease management protocol and standing order guideline for utilization within the present system and method for disease management according to a preferred embodiment of the present invention;

FIG. 10 illustrates an exemplary embodiment of a disease management protocol and standing order guideline for utilization within the present system and method for disease management according to a preferred embodiment of the present invention;

FIGS. 10A-10K illustrate an exemplary embodiment of a scripted assessment for utilization within the present system and method for disease management according to a preferred embodiment of the present invention;

FIG. 11 illustrates the components of a clinical information software system of the present system and method for disease management according to a preferred embodiment of the present invention;

FIG. 12 illustrates the components of a patient care management software system of the present system and method for disease management according to a preferred embodiment of the present invention;

FIG. 13 illustrates the components of a web-based patient care management software system of the present system and method for disease management according to a preferred embodiment of the present invention;

FIG. 13A illustrates the sub-components of a web-based patient care management software system of the present system and method for disease management according to a preferred embodiment of the present invention;

FIG. 13B illustrates the sub-components of a web-based patient care management software system of the present system and method for disease management according to a preferred embodiment of the present invention;

FIG. 13C illustrates the sub-components of a web-based patient care management software system of the present system and method for disease management according to a preferred embodiment of the present invention;

FIG. 13D illustrates the sub-components of a web-based patient care management software system of the present system and method for disease management according to a preferred embodiment of the present invention;

FIG. 13E illustrates the sub-components of a web-based patient care management software system of the present system and method for disease management according to a preferred embodiment of the present invention; and,

FIGS. 14-40 each illustrate an exemplary embodiment of a graphical user interface of a web-based patient care management software system of the present system and method for disease management according to a preferred embodiment of the present invention.

### DETAILED DESCRIPTION OF THE PREFERRED AND SELECTED ALTERNATIVE EMBODIMENTS

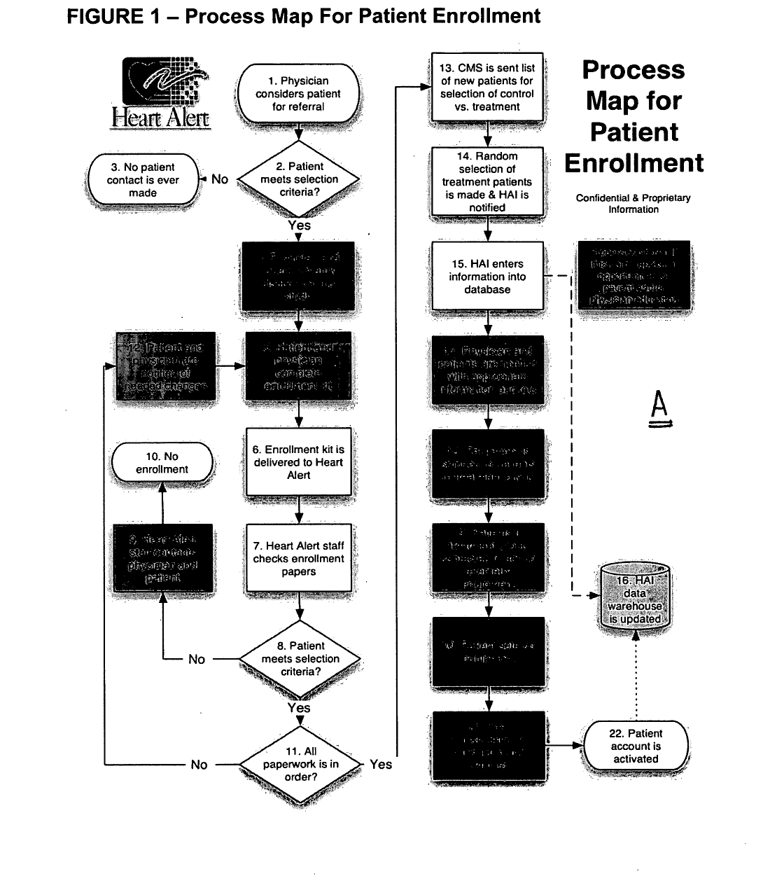

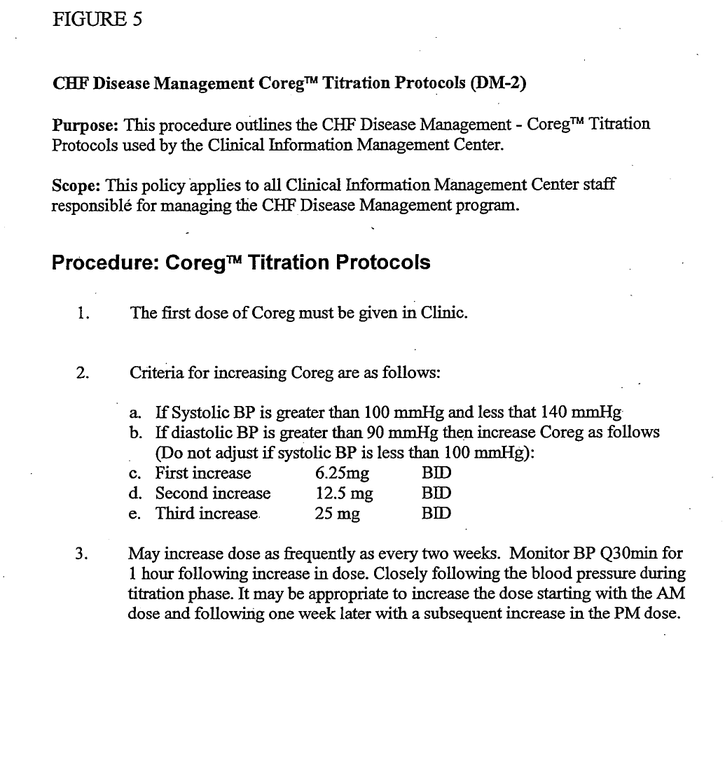

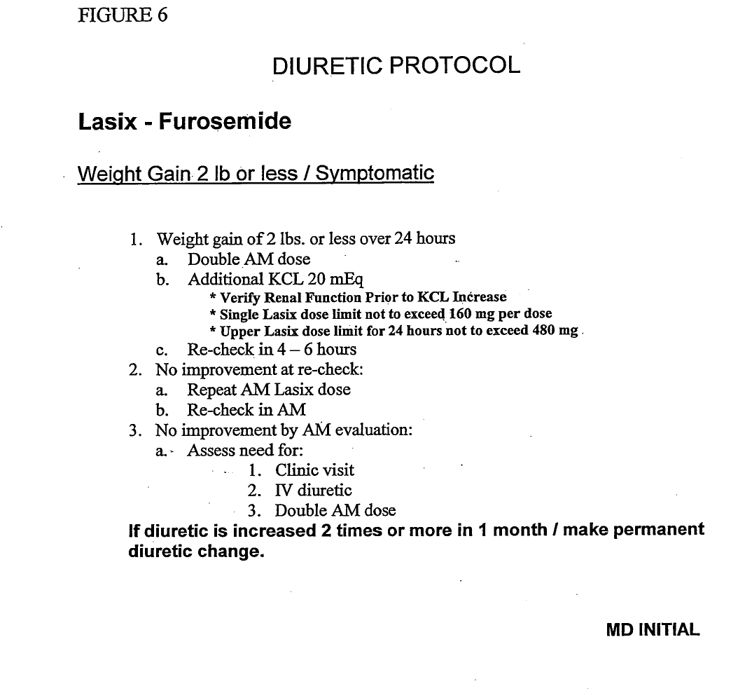

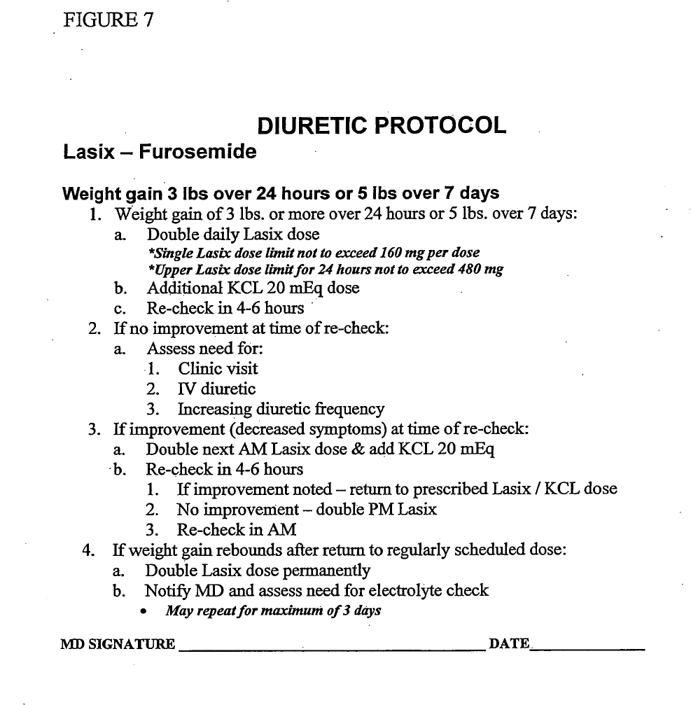

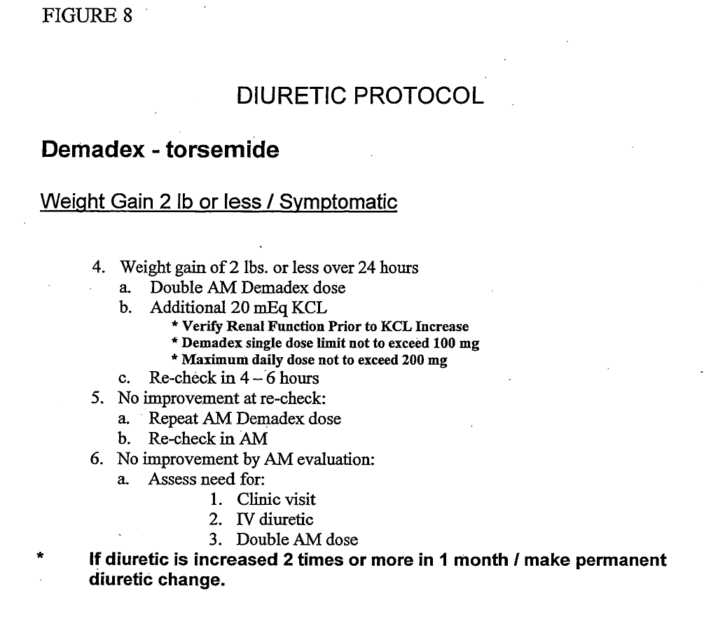

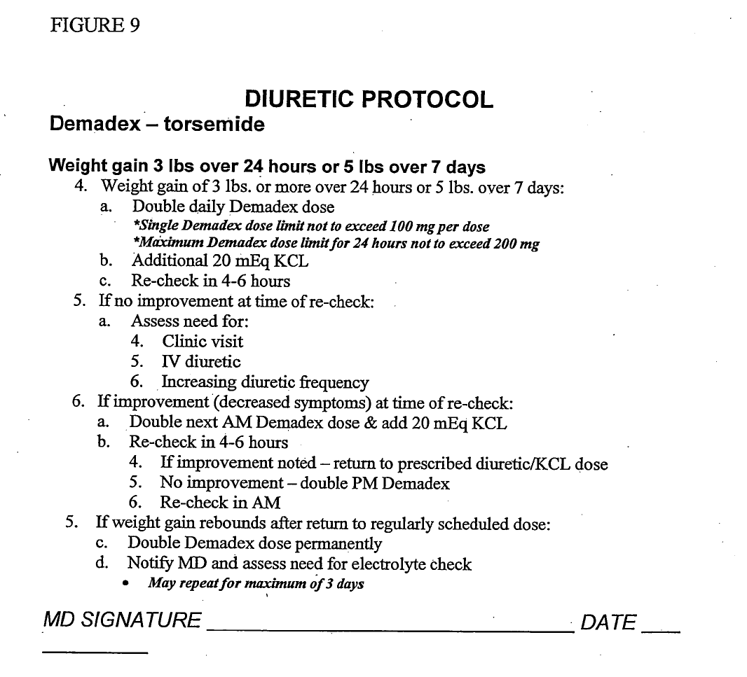

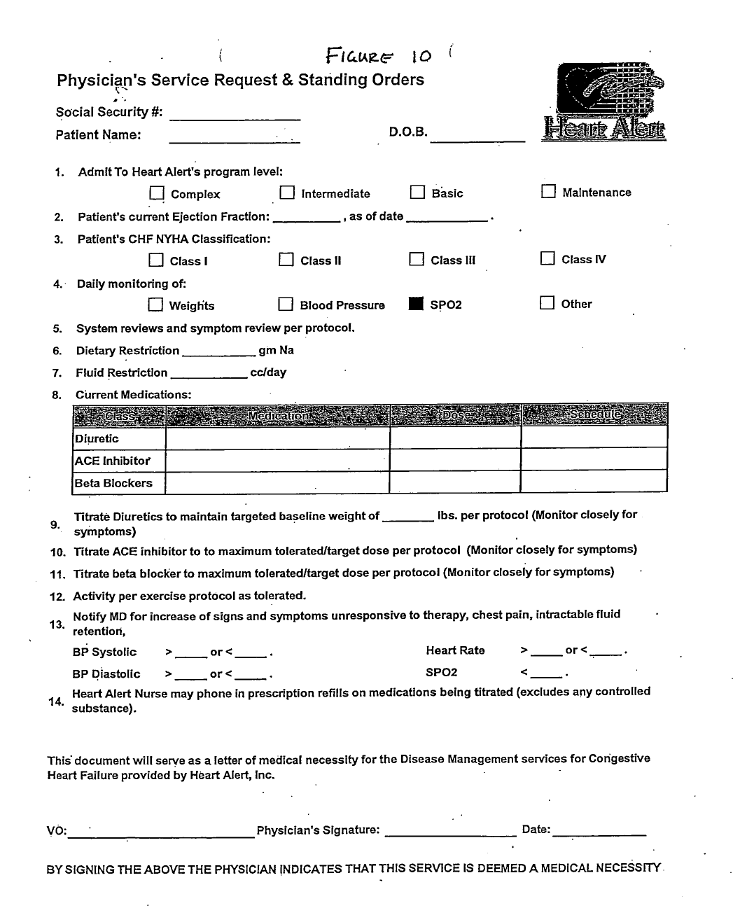

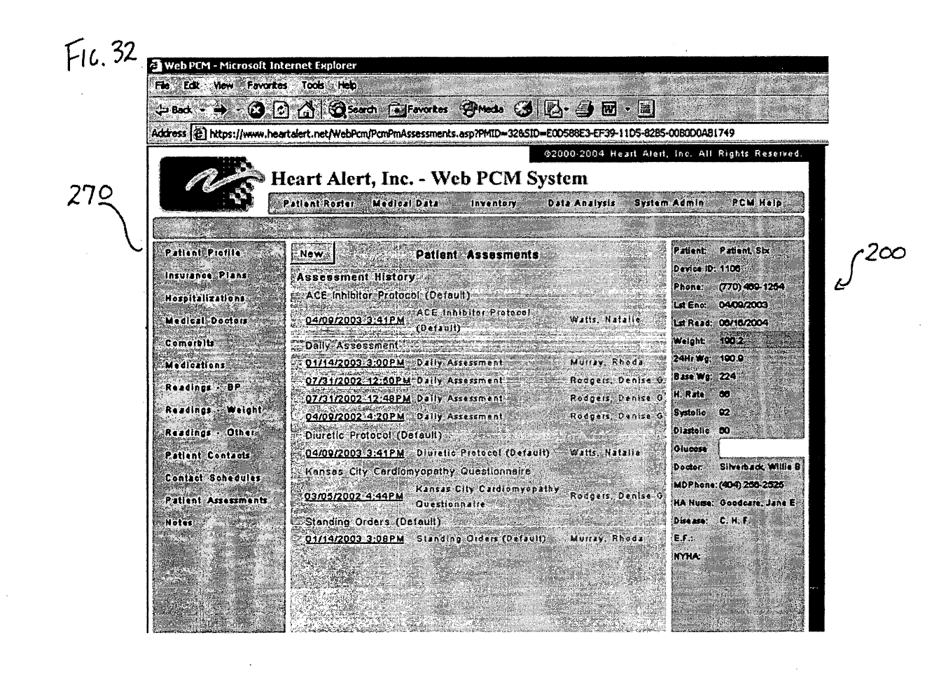

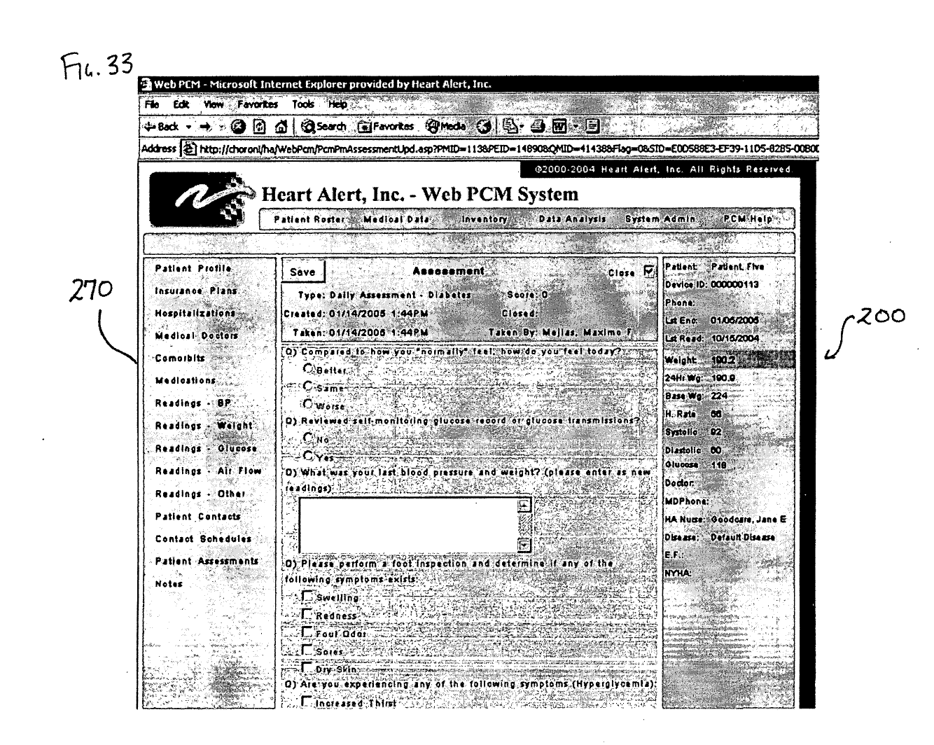

In describing the preferred and selected alternate embodiments of the present invention, as illustrated in FIGS. 1-40, specific terminology is employed for the sake of clarity. The invention, however, is not intended to be limited to the specific terminology so selected, and it is to be understood that each specific element includes all technical equivalents that operate in a similar manner to accomplish similar functions.

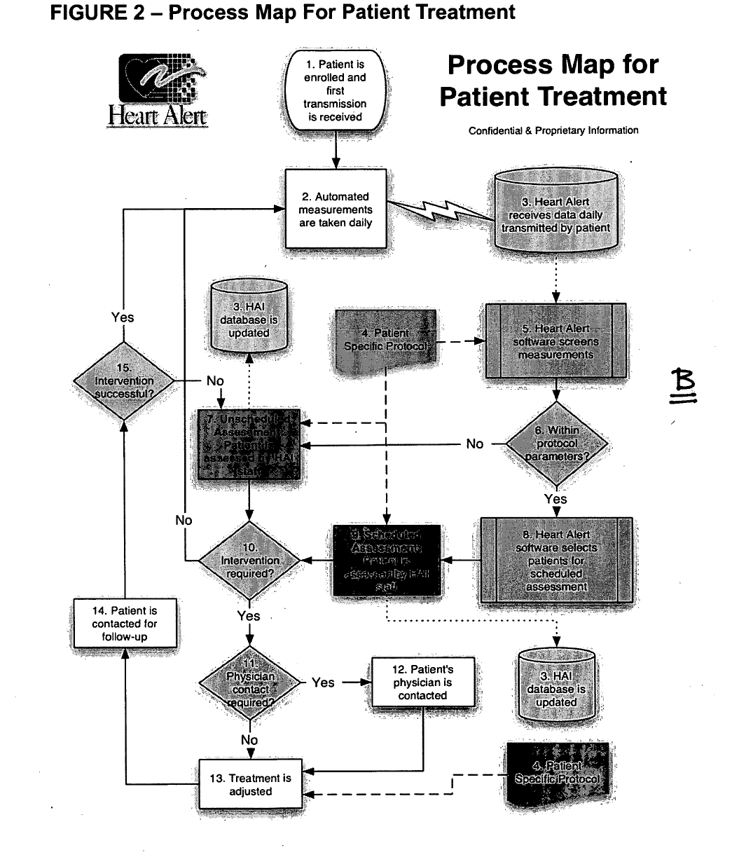

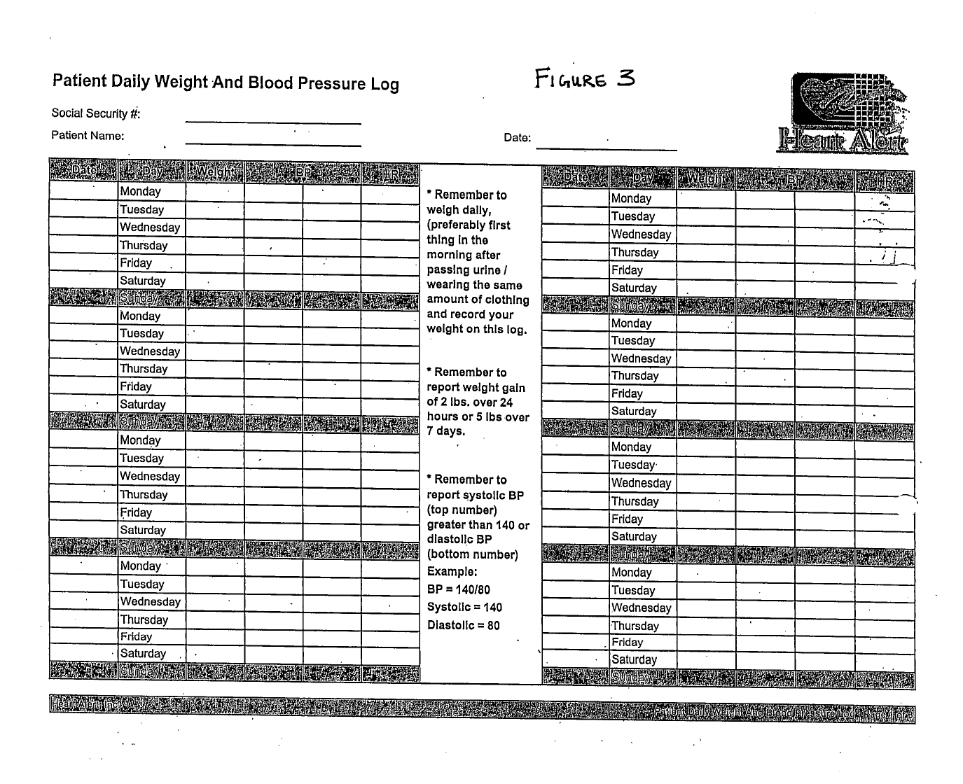

Referring now to FIGS. 1-2, the present invention in a preferred embodiment is a system and method 10 for disease management comprising method for patient enrollment A, method for patient treatment B, and software information system C for managing all aspects of system and method 10.

Referring now more specifically to FIG. 1, patient enrollment method A is preferably initiated via step A1, wherein a treating/participating physician considers his/her patient for referral into system and method 10, as physicians typically have the best understanding of their patients and their patients' disease state. Step A1 preferably contemplates four ways in which a physician can identify a patient for referral: 1) in-clinic visit, 2) hospital discharge, 3) chart review, and 4) referral from another physician.

Following physician referral of the patient into system and method 10, step A2 preferably entails physician evaluation of the patient to determine if the patient is eligible for inclusion into system and method 10. Preferably, and as more fully described below, the inclusion criteria are supplied via a disease management entity utilizing and implementing system and method 10 for delivery of patient care and disease management services. If, based upon the inclusion criteria, the physician determines that the patient is not eligible for inclusion into system and method 10, then the patient is not enrolled (step 3A). If, however, the physician determines the patient is eligible for inclusion into system and method 10, then the physician and patient preferably discuss the process in private, thus avoiding any HIPAA complications, and enabling the patient to make an informed decision as to whether he/she wishes to participate in the present system and method 10 (step A4). If the patient elects to not participate in the process, then the disease management entity will not receive any referral, records, or other patient information.

However, should the patient elect to participate in system and method 10, then the physician and patient preferably complete an enrollment kit (step A5). Preferably, the enrollment kit includes, a patient demographics form (i.e., patient, insurance and physician related information); a Kansas City Cardiomyopathy Questionnaire (i.e., questions that refer to the patient's heart failure and how it may affect his/her life); patient hospitalizations history (i.e., a brief history of hospitalizations, particularly those related to heart failure, or the other chronic diseases, over a selected period); patient medical history (i.e., questions relating to medical conditions that the patient may have been treated for in past years); patient medication profile (i.e., a list of medications the patient is currently taking); a consent to treat form (i.e., official acknowledgement of the patient's enrollment into the present system and method 10); a patient education booklet (i.e., a booklet explaining the disease management program of system and method 10, instructions for setting up and utilizing the electronic monitoring and data transmitting equipment utilized in the present system and method 10 (as more fully described below), recommended diet and exercise plans, and related information about the disease management entity in general); notice of health information practices (i.e., describes how information about the patient may be utilized and disclosed, and how the patient may access same); and, a patient daily weight and blood pressure log (i.e., a patient physiological data log sheet for enabling the patient to track blood pressure and weight readings on a daily basis for facilitating treatment of the patient via patient treatment method B), wherein FIG. 3, preferably illustrates an exemplary embodiment of such a log sheet.

Following review and completion of the enrollment kit by the physician and patient, the enrollment kit is preferably delivered to the disease management entity (step A6). Upon receipt of the completed enrollment kit and paperwork, the disease management entity preferably reviews and checks same for accuracy and completion, and further reassesses the patient's eligibility (step A7). Preferably, step A7 further contemplates that thorough education of the physician and communication with the physician will minimize any future problems, inaccuracies and/or discrepancies associated with completing enrollment kits.

The first check performed by the disease management entity is whether the patient meets the selection criteria for participation within the present system and method 10 (step A8). In the unlikely event that the physician has incorrectly selected a patient who is not eligible for participation within the present system and method 10, the disease management entity preferably immediately contacts the physician and the patient to explain the reasons surrounding such a conclusion (step A9). Step 9 preferably contemplates that immediate feedback to the physician will prevent future incorrect patient referral and selections. If it is definitively determined that the patient cannot be enrolled within the present system and method 10, all patient records and information received by the disease management entity are preferably destroyed (step A10).

The second check preferably performed by the disease management entity is a second determination of completion and thoroughness of all patient paperwork and information (step A11), wherein the patient and physician are notified of any needed changes (step A12).

After satisfaction of completion and thoroughness of all paperwork and patient information, the disease management entity preferably sends a list of new patients to a payor (i.e., Medicare HMOs, HMOs, PPOs, and/or government healthcare providers/payors) for selection of control versus treatment patients, wherein the list is preferably sent to the payor on an agreed upon schedule (step A13). Preferably, the payor is admitted into the present system and method 10 upon contract completion, wherein the payor is preferably assigned a unique identifier that links the payor to the patient. The payor's contract information is preferably entered into the software information system C, along with reporting requirements, outcome specifications, and billing information, as more fully described below.

Preferably, step A14 entails the payor's random selection of treatment patients and control groups, wherein the disease management entity is preferably notified of the selections so made. Thereafter, the disease management entity preferably enters into the database of software information system C all information corresponding to the patients selected for the treatment group (step A15), wherein the information from the patient's associated enrollment papers (including standing orders, patient protocol, prescription drug history, and other important information more fully described) is preferably entered into data warehouse patient tables (step A16) associated with software information system C.

Contemporaneous with steps A14-A16, the disease management entity preferably receives the list of treatment patients and physicians from the payor, wherein the information is also preferably entered into the database of the software information system C. In addition to entry of such physician-patient information, a unique identifier or identification number is also assigned to the patient and physician to enable database linkage between same, and thus the reporting, contacting and retrieval of customized protocols linked to patient records, and outcome tracking.

Similarly, the disease management entity will further admit any ancillary healthcare provider (i.e., hospitals, clinics, homecare providers, and the like) involved in the care of the patient into the database of the software information system C. Preferably, the ancillary healthcare provider will also be assigned a unique identifier linking the ancillary healthcare provider to the patient, wherein all information regarding contacting, reporting and outcome tracking will likewise be entered into software information system C, thereby facilitating implementation of the present system and method 10. The present system and method 10 contemplates that establishment of contact between the disease management entity and the ancillary healthcare provider will function to improve the delivery of care to the patient, wherein the disease management entity will preferably provide reports to the ancillary healthcare provider on a monthly basis, or other designated period of time.

Following entry of all relevant patient, physician, payor and/or ancillary health care provider information into the database of the software information system C, step A17 entails the disease management entity preferably notifying the physician and patients, via appropriate information packets or the like, of the details surrounding the treatment and control groups, and the various processes or actions the physician and patients should expect in the patient treatment method B described below.

Thereafter, patients within the treatment group are preferably shipped (step A18) electronic monitoring and collection equipment for receiving patient physiological data during implementation of the patient treatment method B.

Preferably, step A18 entails the disease management entity generating an equipment request form for retrieving the necessary monitoring equipment from an inventory control division within, or working in association with, the disease management entity. Step A18 further entails assignment of each patient to a management team or “pod” within, or working in association with, the disease management entity, as more fully described below. The inventory control division is preferably responsible for assigning monitoring devices to the patient and entering the device serial numbers into an inventory tracking program of the software information system C, as more fully described below. The inventory control division preferably programs each device for the patient, and thereafter, preferably assembles the monitoring devices, installation instructions and operating instructions for shipment to the patient.

Following shipment of the monitoring devices to the patient, the inventory control division preferably notifies the disease management entity that a new patient is ready for assignment to a pod, and will further notify the pod of shipment and expected delivery date of the monitoring devices to the patients.

Specifically, a pod is a specialized self-contained management team consisting of registered nurses, trained technicians, and medical ancillary staff responsible for patient care and clinical information management. Preferably, each pod is responsible for managing a group of patients, and is designed to facilitate the flow of clinical data throughout the management process of the present system and method 10.

Preferably, upon assignment of the patient to a particular pod, a designated division of the disease management entity preferably provides the pod with all medical forms and information received or developed on behalf of the patient as described above, wherein a tracking program of the present software information system 10 will reflect that such medical information has been requested.

Patients are preferably assigned to a particular pod based on the initial acuity evaluation, pod acuity levels, pod patient load, and managing physician(s). The present system and method 10 preferably attempts to maintain one pod per physician group, wherein each physician is preferably provided with one group of nurses as a contact source; thus, allowing for continuity of care and the development of confidence in physician practice with heart failure programs.

Preferably step A19 entails setting up the telemetry or monitoring equipment delivered to the treatment group/patients, as described above in step A18. The telemetry or monitoring equipment preferably includes a digital sphygmomanometer and a digital scale, wherein each preferably communicate with the pod and/or disease management entity via a modem, or similar global networking communications system, to transmit vital patient physiological data for implementation of the present patient treatment method B, as more fully described below. Preferably, system and method 10 contemplates the specific application of proprietary electronic monitoring equipment available through AEROTEL MEDICAL SYSTEMS, wherein such equipment preferably includes the BPTEL digital sphygmomanometer, and the WEIGHTTEL digital scale; although other suitable electronic monitoring devices capable of transmitting data via modem or similar global networking communications system may be utilized.

Preferably, a medical parameter monitoring system is utilized to enable the pod to record incoming transmissions of patient physiological data via the above-referenced telemetry/monitoring equipment. Preferably, the data transmissions are delivered over available telephone lines, wherein the received transmissions are preferably stored in the appropriate patient file within software information system C. Preferably, each piece of monitoring equipment has a pre-programmed identification code, along with other operational parameters, that is utilized to designate a specific patient and patient location, wherein each identification code and corresponding patient information is preferably entered and stored within software information system C. Accordingly, the identification code of each piece of monitoring equipment is preferably transmitted with each transmission of patient data, and, as such, preferably identifies the calling patient at the associated remote pod station. Preferably, the identification code of each piece of equipment also provides the inventory control division with a means to track the equipment and facilitate the return of same if needed. If the equipment must be returned for repair or upon termination of patient participation within system and method 10, the pod members preferably coordinate with the inventory control division to retrieve the equipment via a suitable mail or courier service, wherein appropriate mail/courier tracking numbers are preferably entered into the patient's file within software information system 10 for providing a tracking mechanism to locate lost equipment or to determine location and estimate time of return of same.

Preferably via software information system C, the medical parameter monitoring system enables the pod and/or disease management entity to monitor and review the patient received and stored data values, to display diagrams and histograms for each patient's records, and to generate printed reports, wherein each such piece of information may be utilized in the implementation of treatment via patient treatment method B described below. Additionally, software/hardware-based receiving stations of the medical parameter monitoring system preferably enable patient records and/or data of multiple patients to be contemporaneously downloaded into specific patient coded files of software information system C; thereby facilitating the contemporaneous evaluation and treatment of multiple patients.

During set up of the patient monitoring equipment within the patient's home or the like, the patient may elect to contact the disease management entity or his/her designated pod for any assistance in setting up the equipment (step A20). Thereafter, a test transmission is preferably conducted and verified to ensure that the monitoring equipment and system are functioning or operating properly (step A21), wherein any issues or problems associated with the telecommunications/monitoring system may be corrected and retested during step A21.

Preferably following verification of operability of the monitoring equipment, step A21 further preferably entails the pod contacting the newly admitted patient to conduct a variety of patient assessments, review the disease management program with the patient, and determine and set patient treatment and outcome goals. Additionally, the pod further educates the patient on a variety of issues, including general heart failure information, patient medications, patient diet plans and goals, patient exercise regimens and goals, and self-care responsibilities.

Furthermore, during contact with the patient, the pod preferably determines the appropriate or applicable disease management level for the new patient, thus further establishing specific patient diagnostic parameters and clinical goals. Specifically, the selected patient management level is preferably determined via the Health-Related Assessment/Kansas City Cardiomyopathy (HRA/KCC) scoring mechanism, wherein the HRA/KCC is preferably designed to determine the degree or acuity of patient heart failure for accurately assessing the applicable New York Heart Association (NYHA) classification of acuity level (i.e., classes or levels I-IV), and thus, the level of patient disease management, as is known within the art. The HRA/KCC requires the patient to answer a series of questions, wherein the type of answer tendered by the patient is numerically quantified based on an associated point system. The total number of points are tallied, and the resulting score compared against specific numerical ranges that correlate to a level of suggested patient management. That is, an HRA/KCC score falling within the range of 76-100 suggests a complex management level; an HRA/KCC score falling within the range of 51-75 suggests an intermediate management level; an HRA/KCC score falling within the range of 26-50 suggests a basic management level; and, an HRA/KCC score falling within the range of 0-25 suggests a maintenance management level.

The complex management level is the highest acuity level, wherein patients require more intensive monitoring, interventions, and follow-up. Patients are typically evaluated every thirty days for progression to a lower acuity level. The diagnostic parameters that typically qualify a patient for the complex management level of care include: NYHA class IV; exacerbation of symptoms managed at home that may require NYHA class IV diueresis; status-post hospital discharge for heart failure exacerbation; conditions where the patient is unstable on current medical regimens; conditions that require actively titrating medications to maximum tolerated therapeutic doses; a patient history of non-compliance with medications, diet and/or fluid limitations; patient has poor support systems; patient has two or more co-morbid conditions; and, a determination that the patient has no knowledge of the disease process. The clinical goals for the complex management level include: patient remains free of symptoms requiring use of NYHA class IV diuretics; patient has no hospitalizations within the past 30 days for heart failure exacerbation; patient is stabilized on current medications with no changes in the past 30 days, patient compliance improves as evidenced by no exacerbation; support systems are identified; stabilized co-morbid conditions; and, the patient has developed an understanding of his/her disease process, and his/her medical regimen.

With regard to the intermediate management level, the diagnostic parameters that typically qualify a patient for such a level of care include: a NYHA Class of III-IV; mild exacerbations requiring increased doses of per oral (PO) diuretics; patient hospitalized for heart failure within the past 30 days; conditions where the patient is stable on current medical regimens; patient receives maximum tolerated doses of medication; patient is compliant with medication, diet and fluid limitation; patient has a fair support network; patient's co-morbid conditions remain stable; and, patient possesses a fair knowledge of his/her disease process. The clinical goals for intermediate management include: improvement of NYHA class by one level; patient remains free of symptoms requiring use of increased doses of PO diuretics; patient is without hospitalizations within the past 90 days; patient is compliant with diet, fluid restrictions and medications; patient and caregiver knowledgeable about disease process and medical regimen; and, patient is without complications of co-morbities.

With regard to the basic management level, the diagnostic parameters that typically qualify a patient for such a level of care include: a NYHA Class of I-III; no exacerbations of heart failure within the past 90 days; no patient hospitalizations in the previous 6 months; conditions where the patient is stable on current medications; conditions where the patient is compliant with all aspects of his/her medical regimen; patient possesses a good support system; patient is stable or has no co-morbidities; and, patient expresses knowledge of his/her disease process. The clinical goals for basic management include: patient remains at NYHA class I or II; patient remains free of symptoms of heart failure for 6 months; patient is without hospital or emergency room visits for 6 months; patient undergoes no changes in medications for 6 months; patient is compliant with his/her medical regimen; patient has a good support network; and, patient has a thorough understanding of his/her disease process.

With regard to the maintenance management level, the diagnostic parameters that typically qualify a patient for such a level of care include: a NYHA Class of I-II; no exacerbations of heart failure in the past 12 months; patient has a through understanding of his/her medical regimen, including medication, diet and fluid limitations; the patient is totally compliant his/her medical regimen; patient is stable on current medications; patient possesses adequate support systems; and, patient has an understanding of his/her disease process. The clinical goals for maintenance management include: patient remains at NYHA class I or II for greater than 12 months; patient is without hospitalizations or emergency room visits for greater than 12 months; patient is able to track weight and blood pressure independently; and, patient and caregiver assume all responsibility for self-care management.

Following a determination of the disease management level pursuant to step A21, and upon successful completion of equipment test transmissions, the patient's account is preferably activated (step A22). The database of software information system C of the disease management entity is further preferably updated (step A16) to show that the patient's account is active and available to the billing system of software information system C. Accordingly, the patient may proceed through patient treatment method B.

Referring now more specifically to FIG. 2, patient treatment method B begins when the first patient physiological data transmission is received by the pod (step B1). Preferably, patients are advised to transmit blood pressure and weight readings first thing in the morning after urinating, but before breakfast and before the patient takes his/her medication. Additionally, the automated measurements of the patient's blood pressure, pulse, and weight are taken daily, and preferably logged or recorded onto the patient's log sheet (step B2), wherein FIG. 3, preferably illustrates an exemplary embodiment of such a log sheet.

Thereafter, the pod preferably receives the daily transmitted data from the patient, wherein the data is preferably reviewed and stored in the data warehouse of the software information system C (step B3). Preferably, software information system C screens the data and measurements, and compares (step B5) same to patient specific protocols and standing orders (step B4) that are codified and stored in the data warehouse of software information system C.

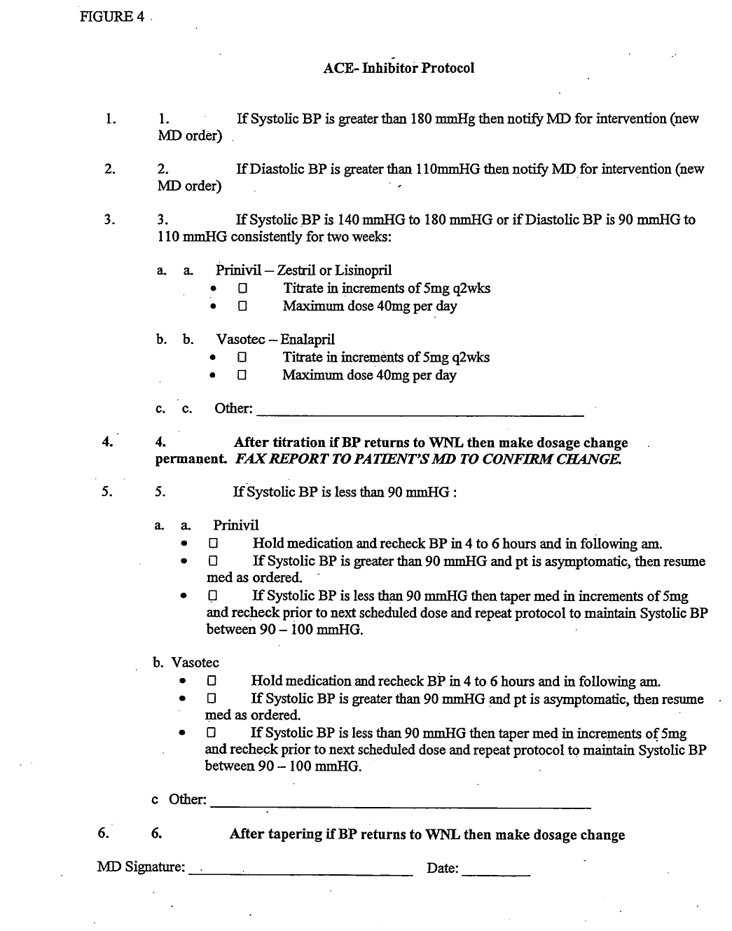

Specifically, and as best illustrated in FIGS. 4-10, protocols and standing orders are preferably treatment guidelines customized for each patient by the managing physician, wherein such protocols are preferably based upon hospital practice, ambulatory clinic practice, physician office practice, consulting with other physicians for co-morbid conditions, ancillary healthcare providers (i.e., home health agencies, disease management entities, pharmacies, hospices, and the like), and wherein the standing orders are preferably based upon published best practice guidelines. Protocols allow the pod to intervene when necessary to prevent further deterioration in condition, relieve symptoms, and prevent patient hospitalizations. The use of protocols preferably alleviates the need for the pod staff to notify the physician for frequently recurring problems, thereby providing the physician with an efficient mechanism to closely follow each patient's condition and/or disease process.

Preferably, step B6 entails the determination of whether the patient's physiological data is within protocol parameters. Specifically, software information system C screens each patient everyday based on the daily transmitted measurements/data in comparison to the patient's protocol. If the measurements are out of the range set by the protocol, the information is preferably flagged on the computer screen of a pod staff member (i.e., preferably a nurse) for review.

Thereafter, an unscheduled patient assessment is made by the pod to determine the reasons why the patient's data is out of protocol range (step B7). For example, if a patient's weight increases, the assessment is geared to the patient's diet and the use of diuretics. The patient encounter is preferably driven by the patient's protocol (step B4) and is codified and stored in the data warehouse (step B3) of software information system C.

More specifically, unscheduled encounters are patient encounters that occur outside of the patient's routine contact with the pod. Such unscheduled encounters can be initiated by the patient for symptoms, or by the pod for transmitted data falling outside set parameters or protocols.

Preferably, step B8 entails software information system C selecting patients for scheduled assessments based upon the patient's current condition. Patients preferably receive scheduled assessments one to three times a week, depending on their current health state. Patients are further preferably evaluated every thirty days for changes to their contact schedule. The NYHA classification, clinical data, diet, and symptomatology are all evaluated before any changes to the contact schedule are made. Should a patient significantly improve in any such category, the patient's contact schedule may be selectively modified, wherein the patient will be reevaluated again in thirty days.

Scheduled encounters are regularly occurring contacts with the patient, wherein the scheduled encounters preferably may be in the form of either (1) transmission of physiologic objective data (i.e., daily weight measurements performed via the digital weight scale, and blood pressure and heart rate measurements performed daily via the digital sphygmomanometer), or (2) collection of subjective data via telephone utilizing internally developed and nationally recognized questionnaires (i.e., health risk assessment/medical history and compliance assessment; assessment questionnaire; Kansas City Cardiomyopathy questionnaire; and, SF-12 and SF-36 satisfaction).

Preferably, each management level has a specific number of scheduled encounters per week or month. Specifically, complex management preferably includes: 2-3 scheduled encounters per week; daily transmission of objective physiologic parameters; daily review and trending of transmitted data; and, unscheduled encounters as warranted from transmitted objective data outside parameters, or activated via patient complaint. Intermediate management preferably includes: one scheduled encounter per week; daily transmission of objective physiologic parameters; daily review and trending of transmitted data; and, unscheduled encounters as warranted from transmitted objective data outside parameters, or activated via patient complaint. Basic management preferably includes: one scheduled encounter every two weeks; daily transmission of objective physiologic parameters; daily review and trending of transmitted data; and, unscheduled encounters as warranted. Maintenance management preferably includes: one time per month encounters; daily transmission of physiologic parameters; daily review and trending of transmitted data; and, unscheduled encounters as warranted.

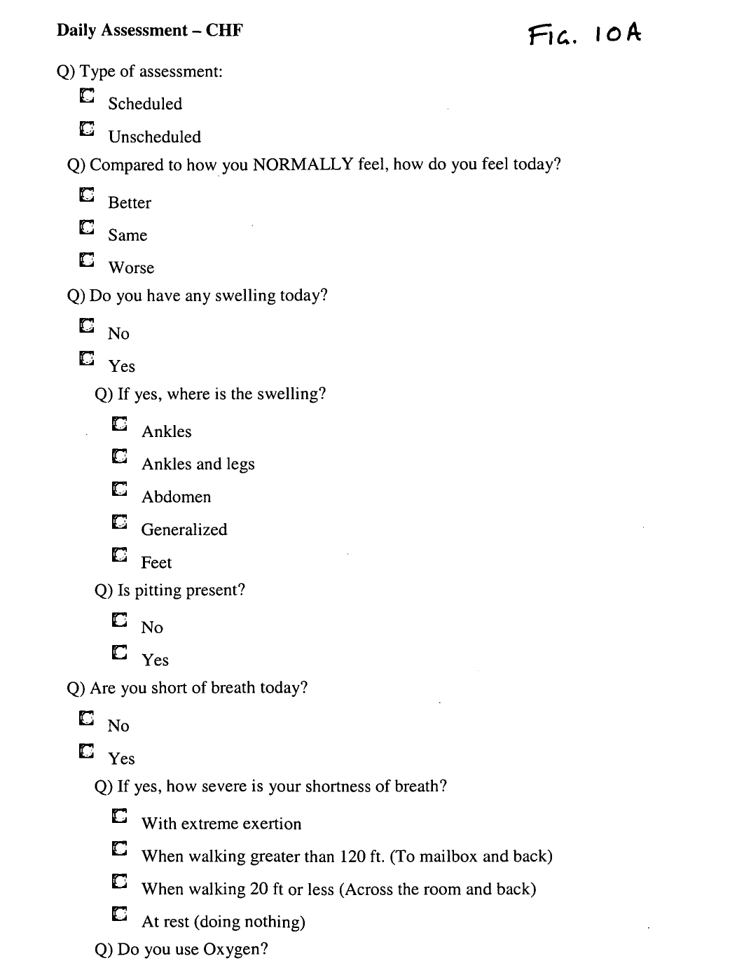

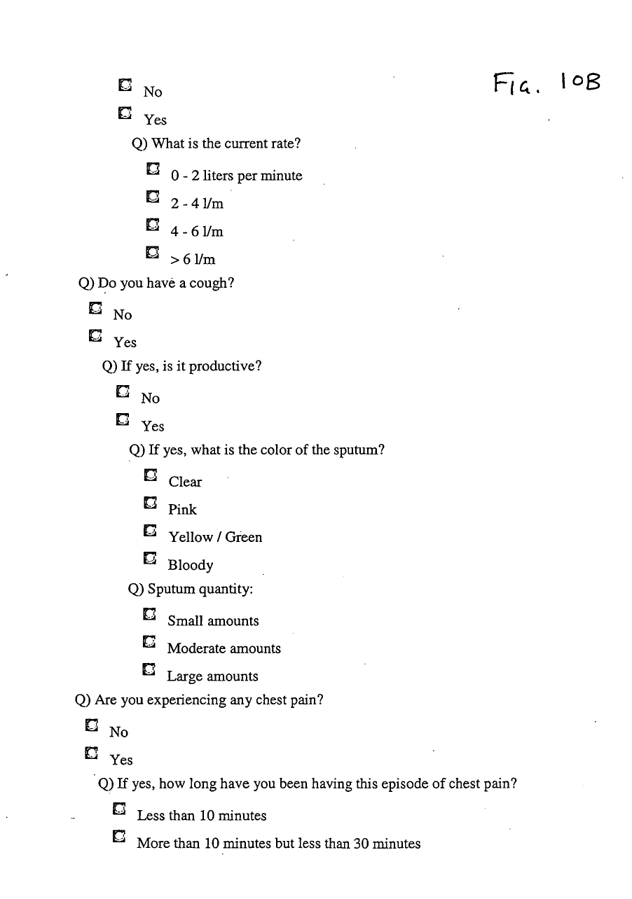

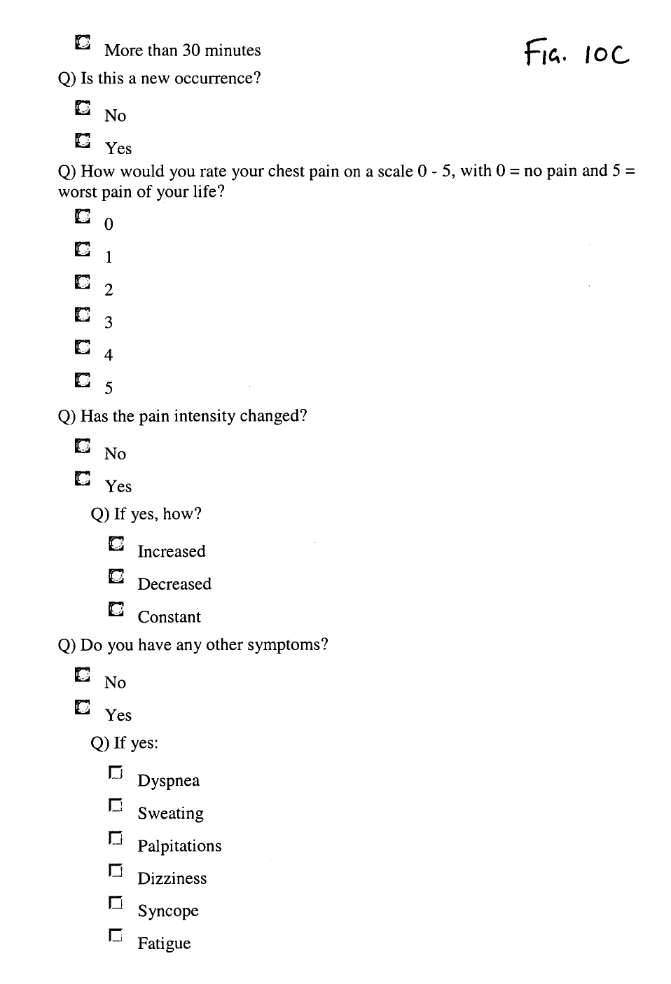

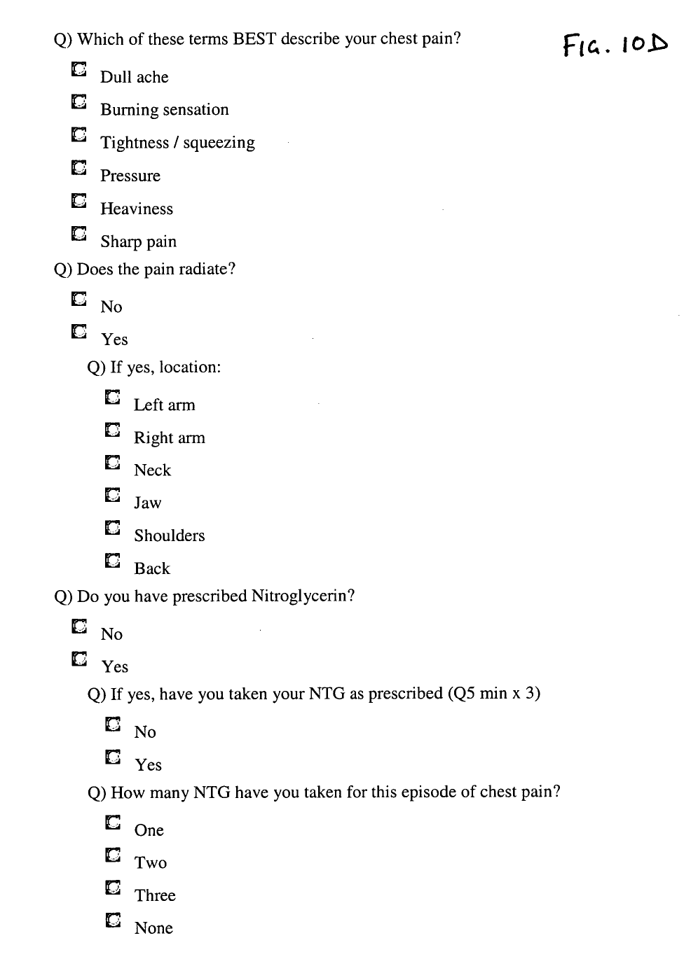

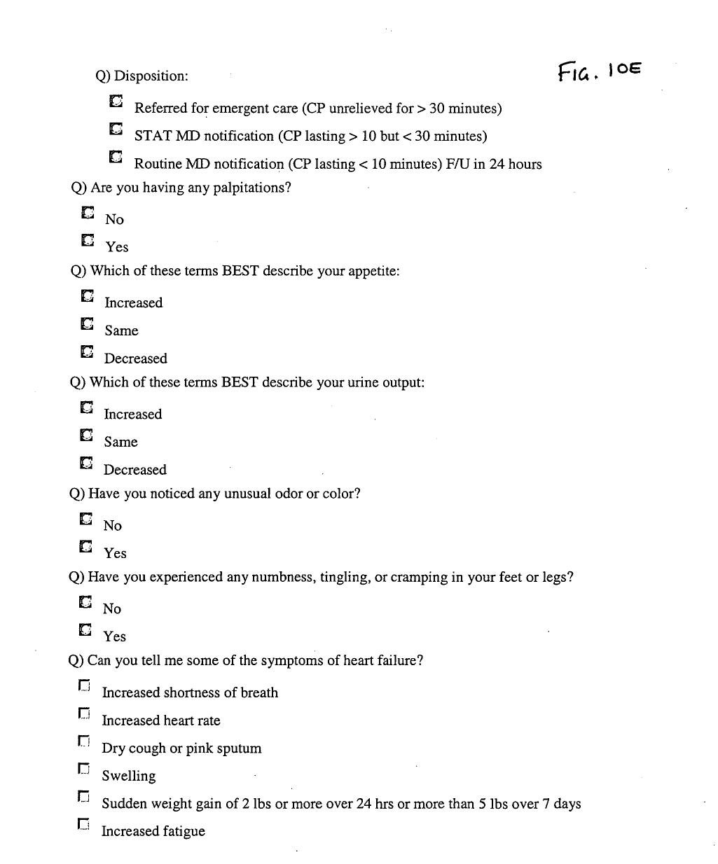

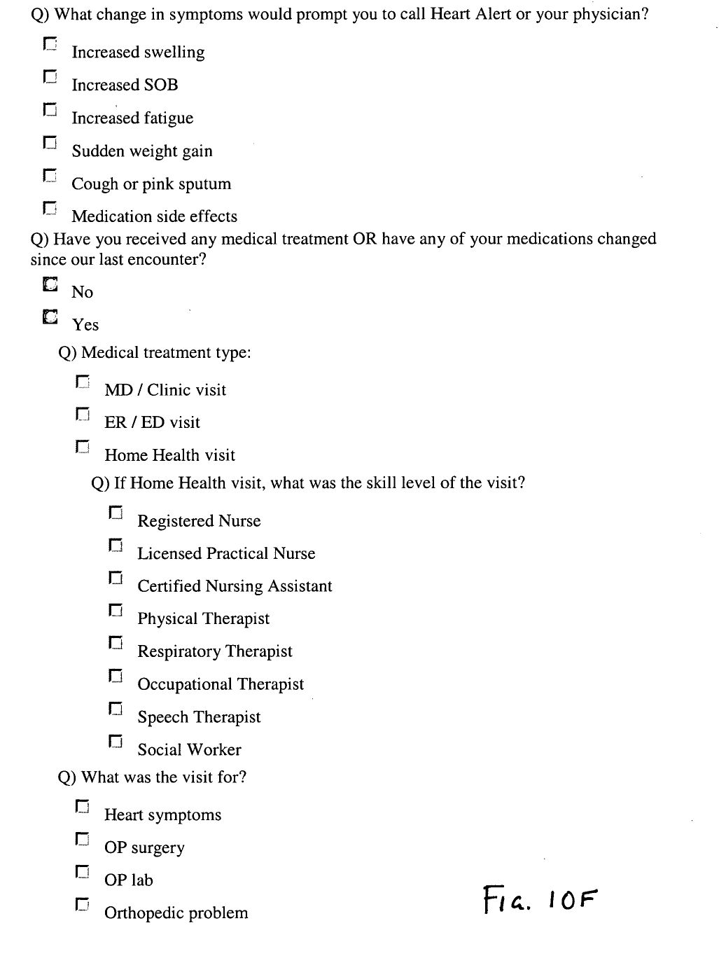

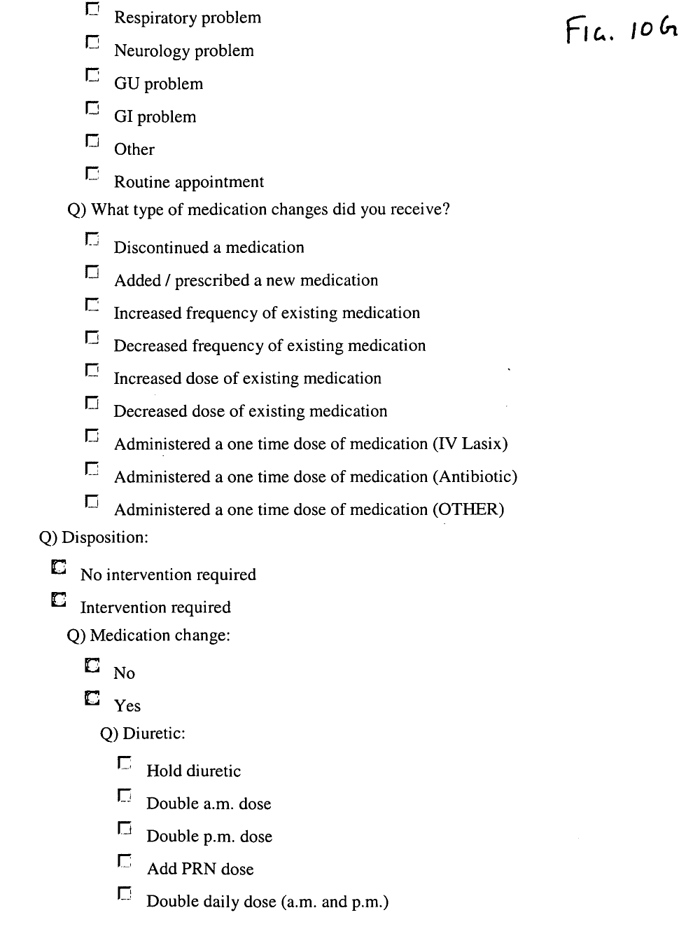

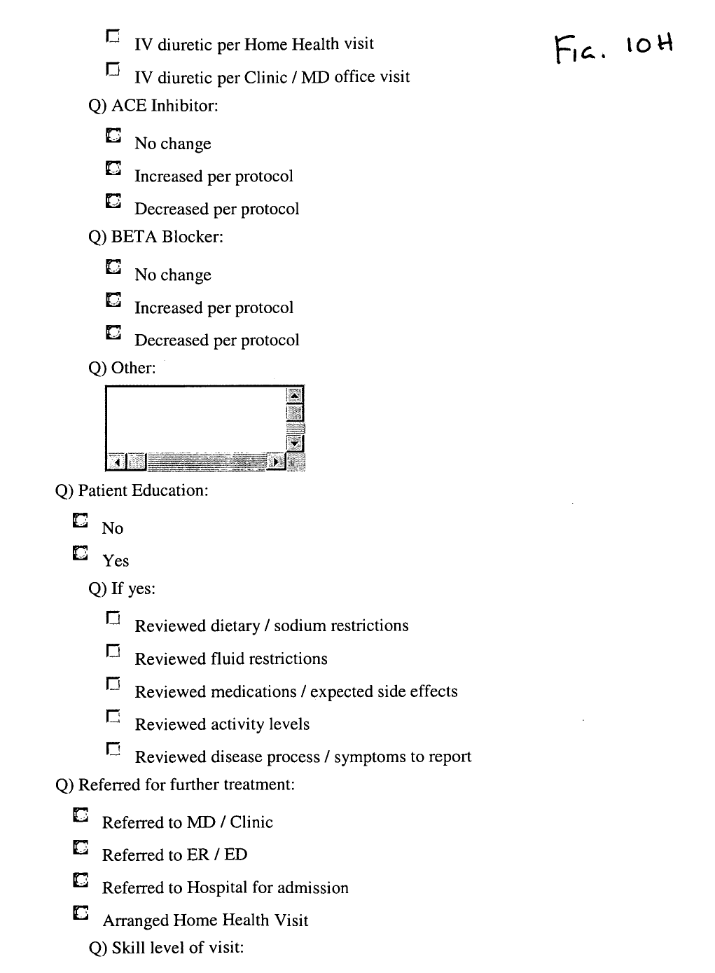

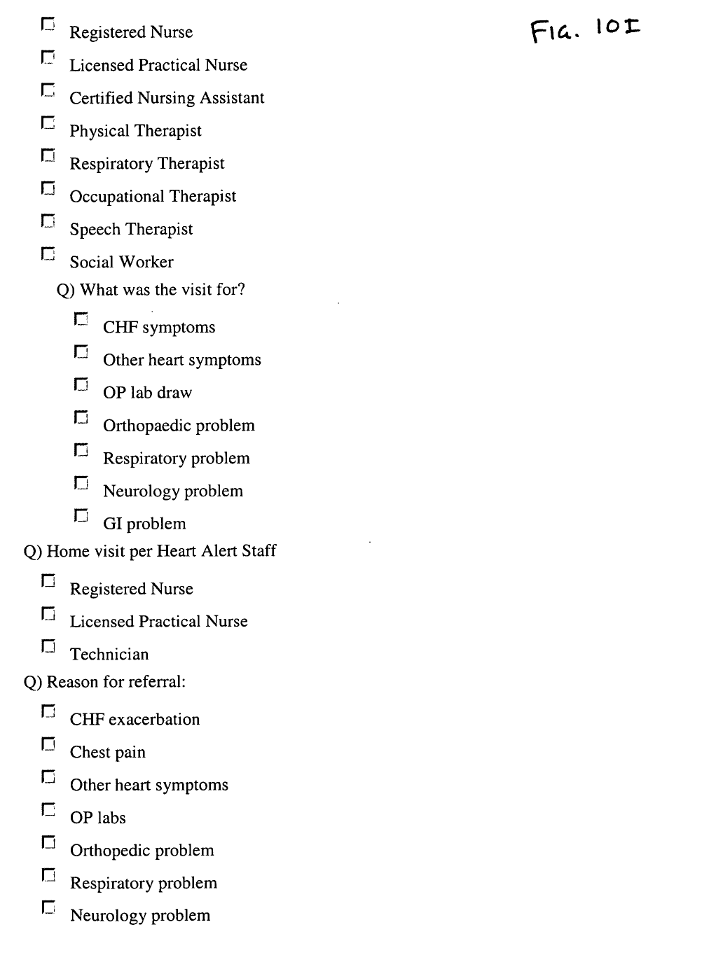

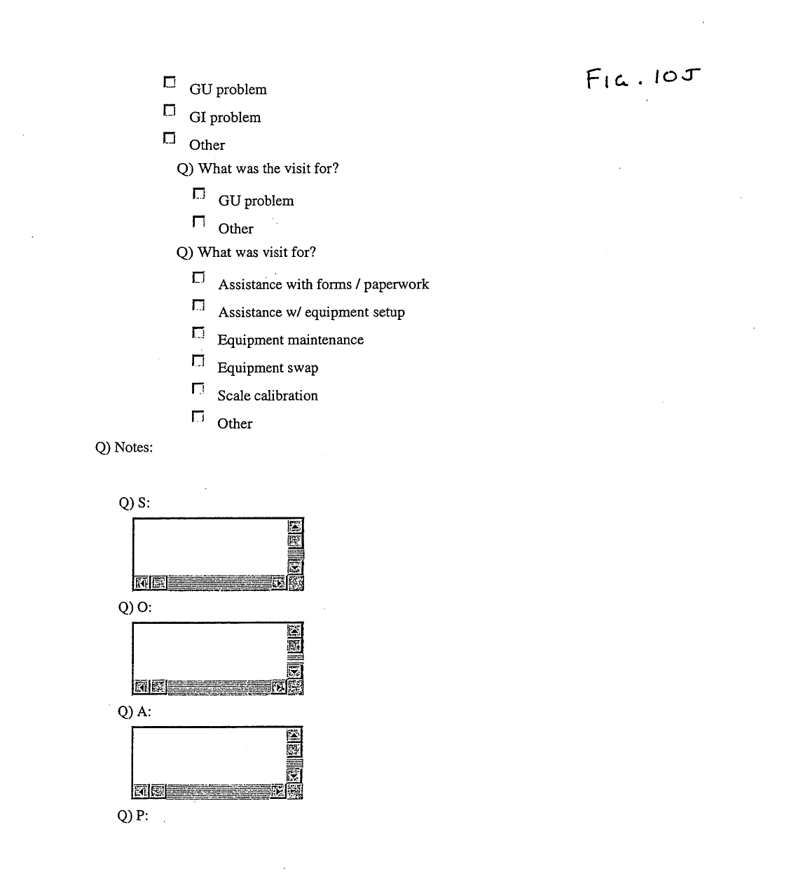

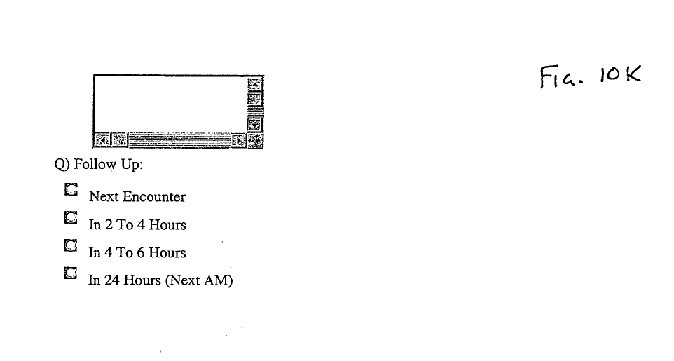

Preferably, step B9 entails the actual scheduled assessment of the patient by the pod staff. Preferably, scheduled assessment is driven by the patient specific protocol, wherein the scheduled assessment is precisely scripted (see FIGS. 10A-10K for an exemplary embodiment of such scripted assessments), and the results stored in the data warehouse of software information system C. Utilizing such stored assessments, a patient's entire history with the disease management entity is available to authorized personnel, including the patient's physician.

Preferably, all patients receive the same level of service from the pod, but are evaluated and classified into preferably one of three levels for scheduled nurse/pod calls. However, any patient may receive an unscheduled call at any time, based upon the health indicators received daily from the electronic telemetry equipment and system.

Preferably, a critical decision made by the pod staff during an assessment, scheduled or unscheduled, is whether any intervention in the patient's treatment is required (step B10). If so, then a determination must be made as to whether the patient's physician should be contacted. If no intervention is required, however, the assessment is complete and the process begins anew the next day with the automated measurements taken daily.

Specifically, the pod/disease management entity preferably utilizes the standing orders and protocols to intervene when necessary in the course of the disease management process and patient treatment method B, in general. Preferably, the pod intervenes when necessary to prevent re-hospitalizations of the patient for exacerbation of heart failure (or other chronic disease).

During any assessment, the protocol or a pod staff member may be required to determine or consider whether the patient's physician should be contacted (step B11). If an adjustment to the patient's treatment is allowed by the protocol, but no physician contact is needed, then the adjustment is made by the nurse. For example, the protocol may allow for a small increase in the patient's diuretic medication.

The physician will be contacted as a result of either a scheduled or unscheduled assessment, or ongoing oversight of the patient's disease state (step B12). The patient's physician plays a key role in the treatment regimen. In addition to patient monitoring, the physician works directly with the pod staff to optimize the patient's treatment. The physician further preferably has secure online access to his patient's records via software information system C, twenty-four hours a day, seven days a week.

Based upon either step B11 or B12, if required, the patient's treatment regimen is changed based on the instructions in the protocol or the direct orders of the physician (step B13). Any and all changes are preferably documented in the data warehouse of software information system C. Once the treatment is changed, the process repeats itself the following day.

Thereafter, if there were any changes to the patient's treatment (i.e., an intervention), a follow-up contact is scheduled, usually the same day (step B14). The follow-up contact preferably ensures the change had the intended effect.

If the change in treatment (i.e., intervention) was successful, then the process repeats the next day via the automatic transmission of new data from the patient (step B15). If the intervention was not successful, then the patient preferably undergoes another unscheduled assessment, and further changes are made and/or the physician is contacted.

Patient education is preferably an ongoing process throughout patient treatment method B. Preferably, each patient will have a patient education book as part of the admission packet, wherein the book will be utilized throughout the initial admission process and ongoing throughout patient treatment method B. Periodically throughout the course of management, the patient will be sent additional educational literature to include in the education book.

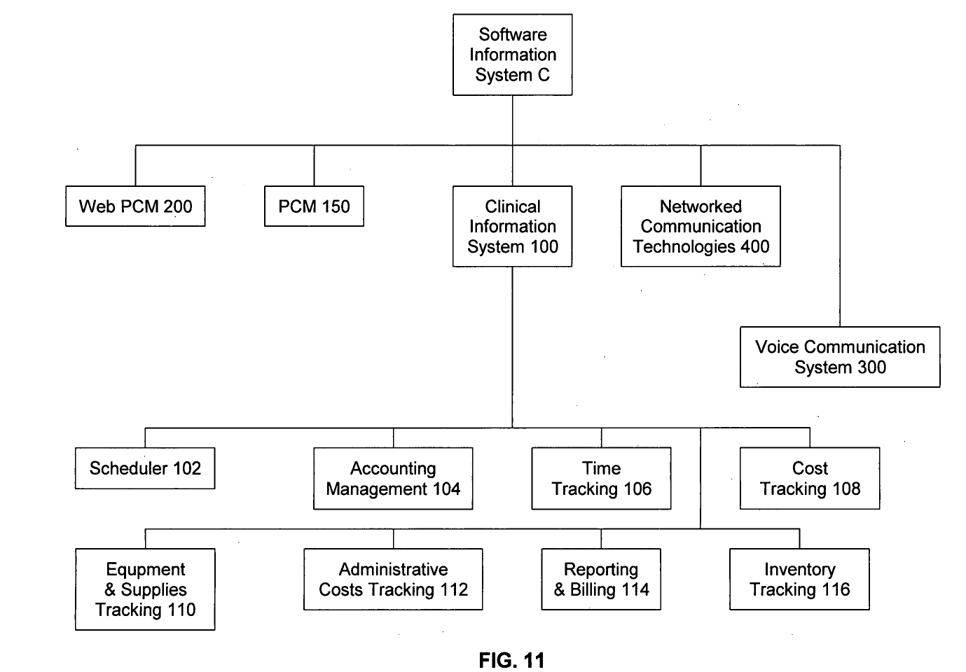

Referring now more specifically to FIG. 11, system and method 10 preferably includes software information system C, wherein software information system C comprises a clinical information system 100, patient clinical management software (PCM) 150, web-based version 200 of patient clinical management software 150, voice communications system 300, and multiple software packages integrated into a network. Broadly, software information system C preferably functions to manage all aspects of system and method 10.

Clinical information system 100 operates as a single data entry point that communicates clinical, financial, billing, and sales data throughout system 10. Accordingly, clinical information system 100 preferably includes a scheduler 102 that is utilized to schedule encounters/assessments based upon management levels. Preferably, clinical information system 100 enables the pod to: select management level for schedule generation; search open dates and times for scheduled interventions; generate a daily call log of scheduled encounters; schedule events which occur less frequently (i.e. monthly surveys, report times, and the like); provide reminders for events; and, track actual encounter times.

Clinical information system 100 further preferably includes accounting management system 104 to assist in cost tracking of time, equipment and supplies, and administrative costs. Specifically, with regard to cost tracking, when a patient is enrolled into the present system and method 10, an ID number assigned to the patient will be utilized to track the associated cost of management. The ID number is a series of numerical codes specific to each patient. The number consists of a patient identifier, the attending physician identifier, and payor identifier, and preferably enables cost tracking for all three identifiers.

The most critical cost-tracking factor of system and method 10 is time. Accordingly, clinical information system 100 of software information system C preferably monitors the time 106 spent on each patient interaction and distinguishes the nurse, technician, or doctor working with the patient. Based on the salary of the nurse, technician, or doctor, and the time of interaction, clinical information system 100 tracks cost 108.

Preferably, equipment and supplies tracking 110 of clinical information system 100 tracks and inventories all equipment and supplies, including shipping and handling costs, via ID numbers, wherein the utilization of equipment and supplies can be documented, and thus the cost per patient, attending physician, and payor recorded.

Clinical information system 100 further preferably tracks the internal or administrative costs 112 of patient management within the disease management entity utilizing/implementing system and method 10.

In addition to cost tracking, clinical information system 100 further preferably provides reporting and billing functions 114. Specifically, external reporting of actual costs to the payor are preferably conducted through software information system C. With regard to billing, a bill for services is generated on a monthly basis and sent to the payor, wherein software information system C generates a detailed bill utilizing program type and activation date to calculate the appropriate fees to charge. Additionally, those patients enrolled in the program during the month have an activation fee charged and the monthly rate prorated.

Preferably, detailed cost reports, are generated for each individual patient, for an attending physician with multiple patients on the program, and for a payor with multiple patients on the program. The reports are preferably generated monthly, quarterly and annually. If applicable, actual results may be compared to established projections. Additionally, internal reports, including financial statements, equipment utilization reports, time utilization reports, and supply utilization reports, may also be generated via reporting and billing functions 114 of clinical information system 100.

Clinical information system 100 further preferably includes an inventory tracking software program 116, wherein inventory program 116 is preferably in an access database format. Preferably, once the third-party manufacturer monitoring equipment arrives at the disease management entity's location, it is preferably taken to the inventory control division for logging in. Preferably, logging the equipment occurs via a manual log sheet to which a packing slip is attached, wherein the equipment is further logged or entered into inventory tracking software 116 via the equipment serial number. Preferably, the digital scale and the digital sphygmomanometer will each have unique serial numbers that will be enter into the computer system that will allow the complete tracking history of that individual device/equipment piece. Inventory tracking software program 116 preferably further enables the tracking of the device by state, salesman, account, physician, manufacturer, and status of the device. Once the devices have been entered into the inventory tracking software program, each device is available to be “checked-out” to a patient, account or salesman. All documentation and log sheets are preferably recorded in the inventory department by date, thereby facilitating overall tracking, tracing, return and/retrieval of each device.

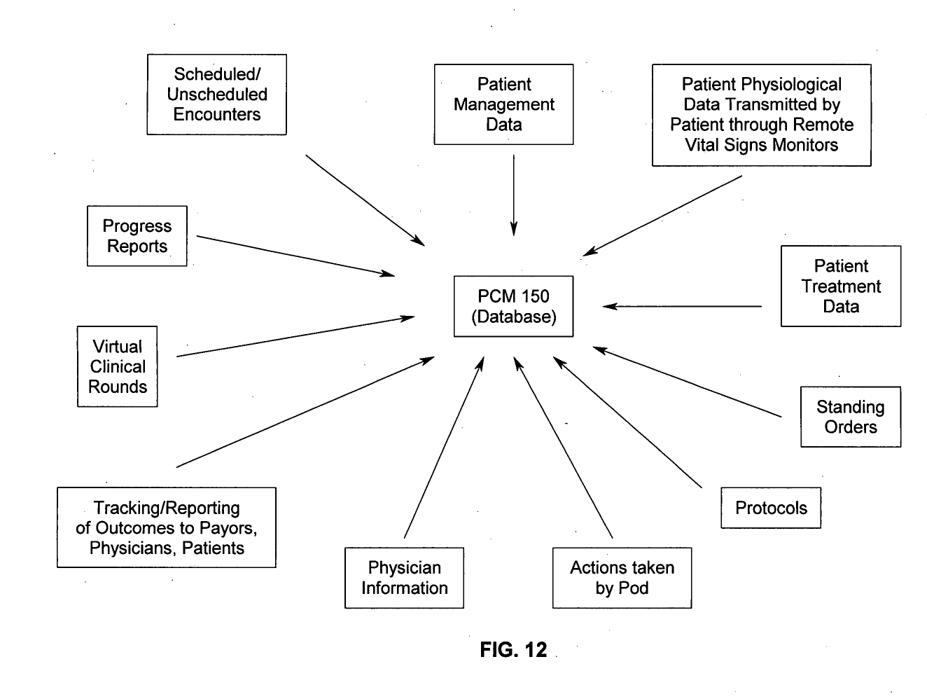

Referring now more specifically to FIG. 12, in addition to clinical information system 100 of software information system C as described hereinabove, software information system C further preferably comprises patient clinical management software (PCM) 150. Preferably, PCM 150 is the central data collection point for patient management and data collection for system 10. That is, PCM 150 preferably functions as the database or data warehouse of software information system C for collecting, storing, analyzing, and enabling the retrieval of patient management data, all physiological data transmitted by the patient through the remote vital signs monitors, and all resulting treatments, protocols, and actions taken by a pod and/or physician with respect to same). PCM 150 is also preferably utilized to track and report outcomes to payors, physicians, and patients. PCM 150 preferably includes Internet/intranet access through an encrypted and firewall protected environment that will allow third party payors and physicians to perform “virtual” clinical rounds on each patients. As patients become more “Internet” capable, each patient will be able to complete monthly satisfaction and progress questionnaires online.

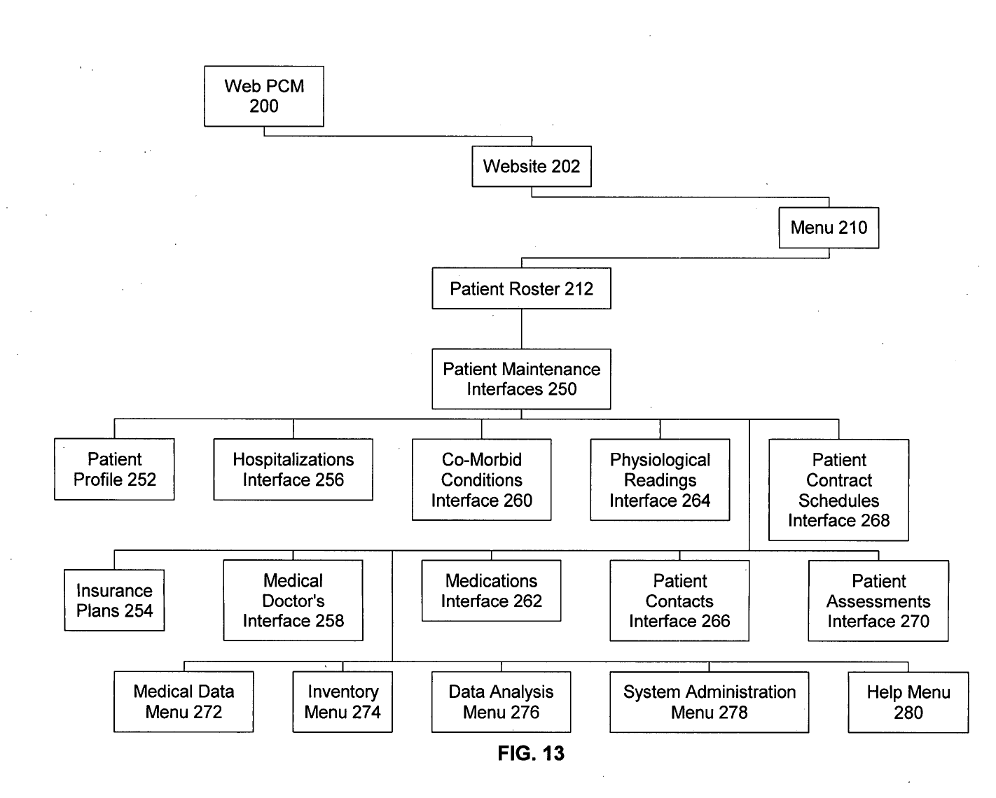

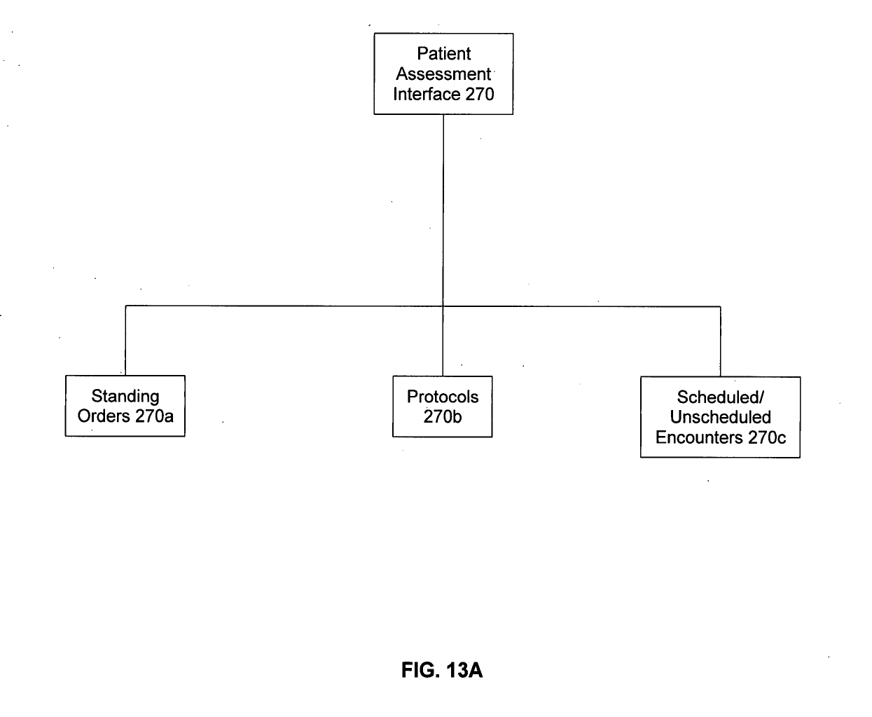

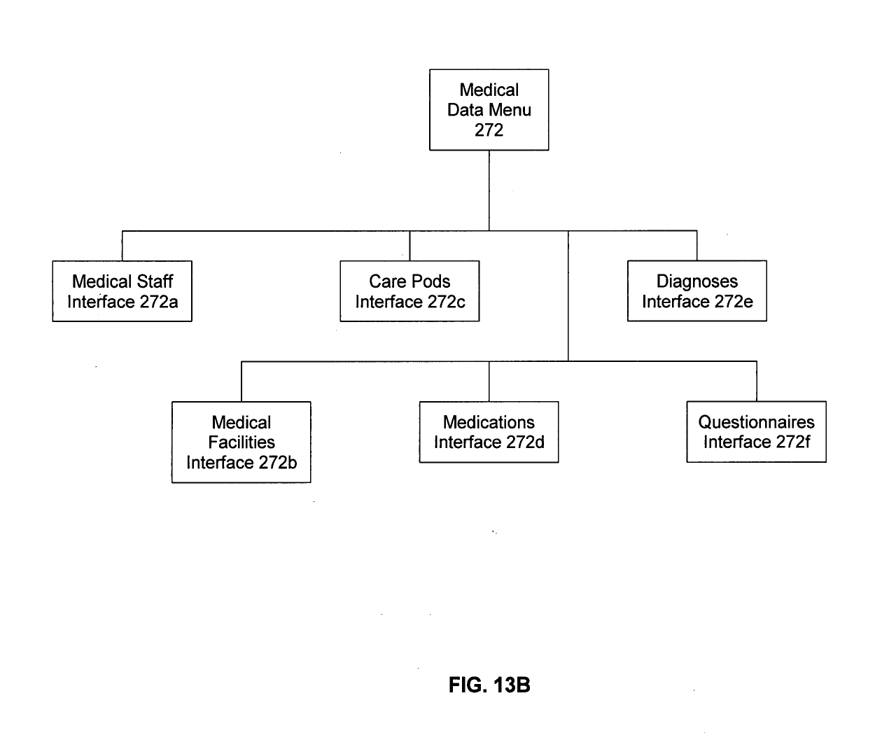

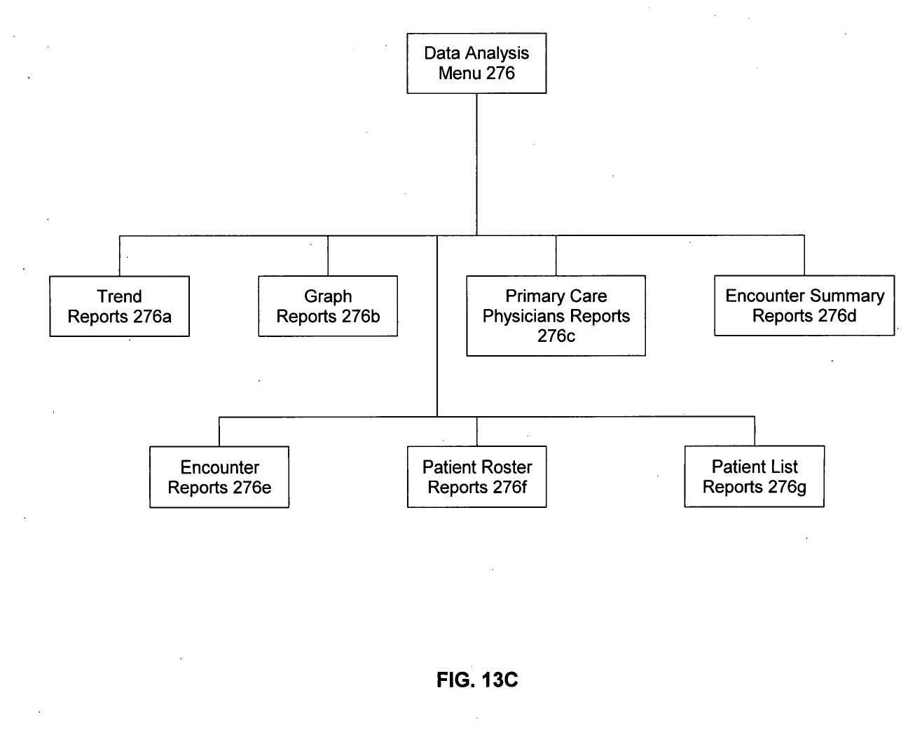

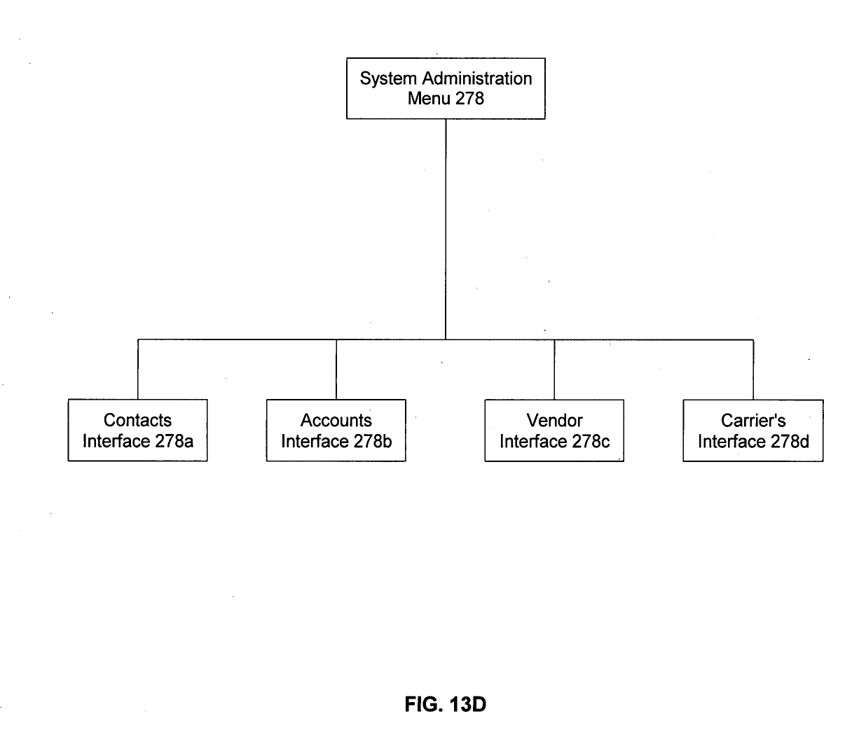

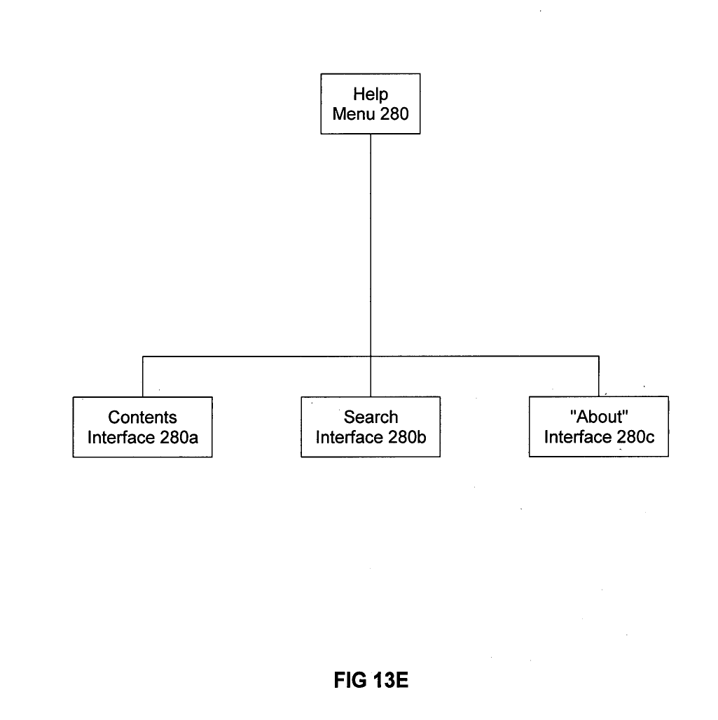

As generally illustrated in FIGS. 13-40, PCM 150 is preferably further provided as a web-based version (Web-PCM) 200, wherein Web-PCM 200 comprises the features and advantages of PCM 150 as described hereinabove, but is conveniently accessible through a global networking system (i.e., World Wide Web, or the like). User access to Web-PCM 200 is preferably restricted to the disease management medical professional team, member physicians and enrolled patients, with each group of users accessing a distinct interface. Moreover, access to Web-PCM 200 is preferably monitored, logged and audited as an additional layer of security. Web-PCM 200 further preferably provides multiple reporting options, including web-based access for patients and physicians, and periodic reports in either hardcopy and/or electronic-mail format delivered to the patient's physicians.

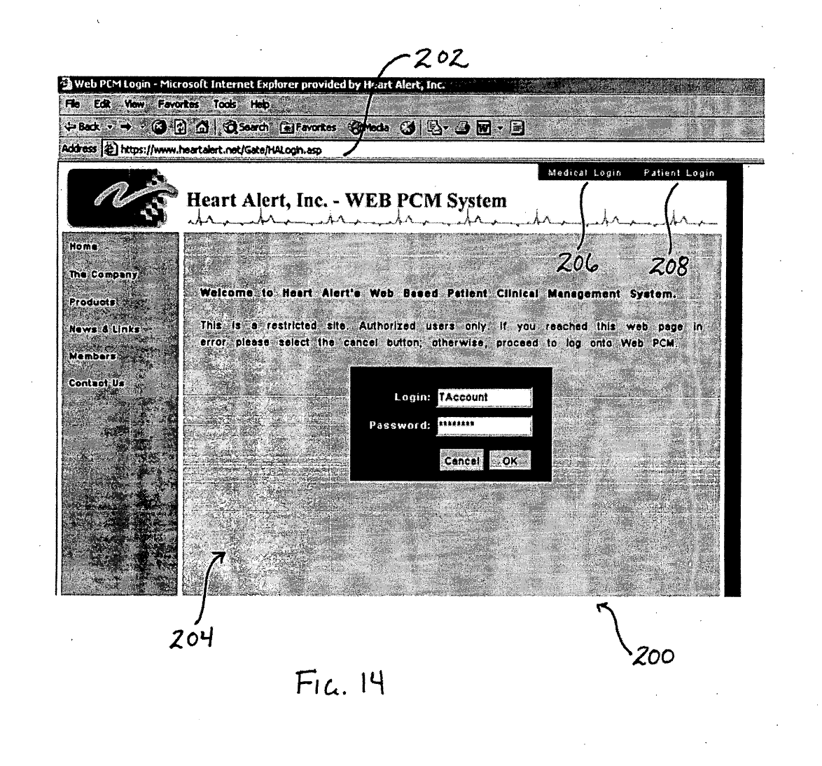

More specifically, and with combined reference now to FIGS. 13 and 14, users may access Web-PCM 200 through an associated website 202, wherein home page 204 of Web-PCM 200 preferably offers portals to enter Web-PCM 200: medical login portal 206 for doctors and nurses, and patient login portal 208 for patients. Upon selecting the appropriate portal, users will be re-directed to a logon screen for authentication. Only authorized users will be granted access to Web-PCM 200, preferably via login and password identification.

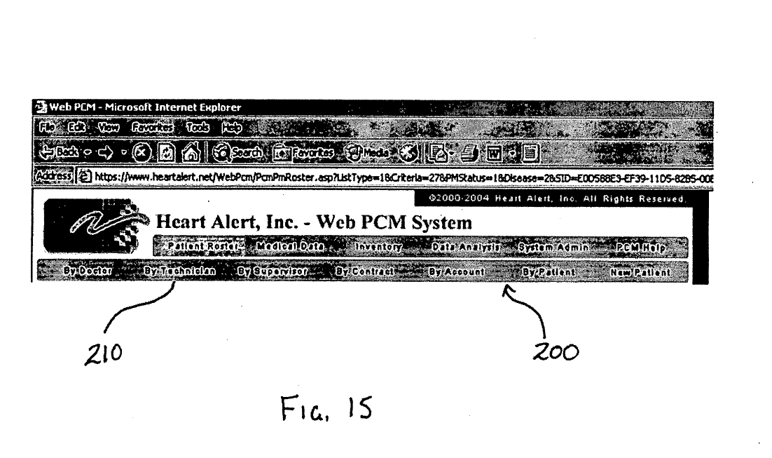

Upon entering Web-PCM 200, users will have access to all system functions through menu 210 of Web-PCM 200, as best illustrated in FIG. 15. Although detailed more fully hereinbelow, menu 210, and the various interfaces accessible therethrough, present users with the following general functional areas: (1) a patient roster, which provides access to interfaces utilized to manage patient data, and further enables users to generate lists of patients according to user specified criteria (i.e., rosters for active and/or inactive patients for a specific account, contracts, nurses, referring doctors, or by patient name); (2) medical data, which provides user access to interfaces utilized to manage system-wide medical data, such as medications, hospital and facilities, and doctor demographics; (3) inventory, which provides user access to interfaces utilized to manage telemetric equipment assigned to patients; (4) data analysis, which provides user access to reporting tools; (5) system administration, which provides user access to interfaces utilized to manage system-wide administrative data, such as accounts, contracts, vendors and users; and, (6) help, which provides user access to helpful information for operation and/or navigation through Web-PCM 200.

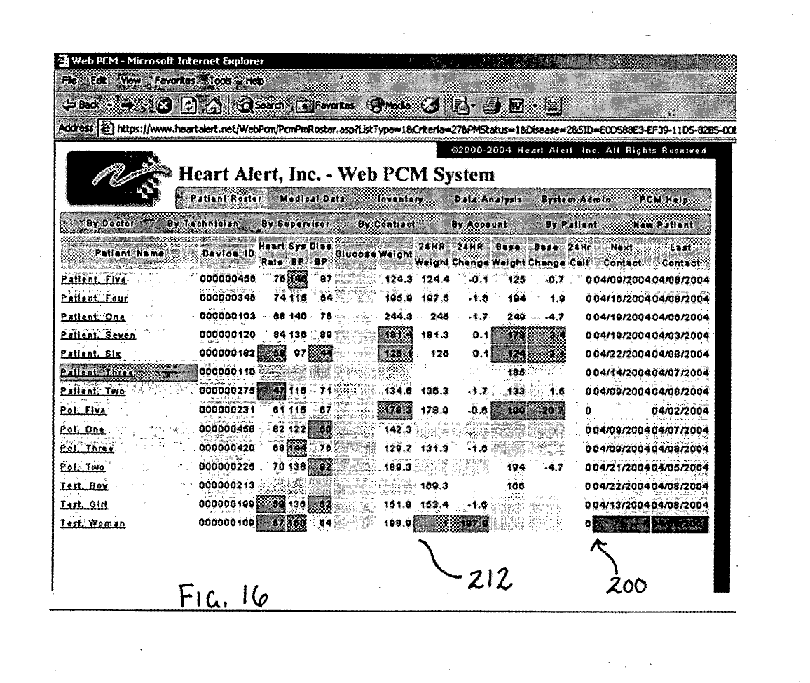

Specifically, and with combined reference now to FIGS. 13 and 16, patient roster interface 212 allows users to generate lists of patients according to user specified criteria. Users can generate rosters for active and/or inactive patients for a specific account, contract, nurse, referring doctor, or by patient name. Patient roster 212 returns a list of patients and displays valuable medical data on the selected patients. Patient roster 212 further displays the patient's daily telemetric readings, any significant change from base readings, and the dates of the last and next contacts. Additionally, patient roster 212 comprises visual alerts to highlight readings that fall outside “normal” parameters, as well as to indicate any pending contacts, wherein the following alerts are preferably available: (1) red reading alerts indicate readings that fall outside “normal” values; (2) yellow reading alerts indicate missing daily values; (3) yellow contact alerts indicate pending contacts; (4) blue contact alerts indicate completed, successful contacts; and, (5) red contact alerts indicate contacts that were attempted but failed because the patient could not be reached.

Patient roster interface 212 further allows users to access all medical data available on each patient by selecting the patient from the roster or list and entering patient maintenance interfaces 250.

Patient maintenance interfaces 250 allow users to access patient demographics, hospitalization history, insurance coverage, medication data, co-morbid conditions, physiological readings, patient contacts, standing orders, assessment and encounters, and any notes entered on the patient. Essentially, patient maintenance interfaces 250 offer a complete picture of the patient's medical state, as well as the care plan for the patient.

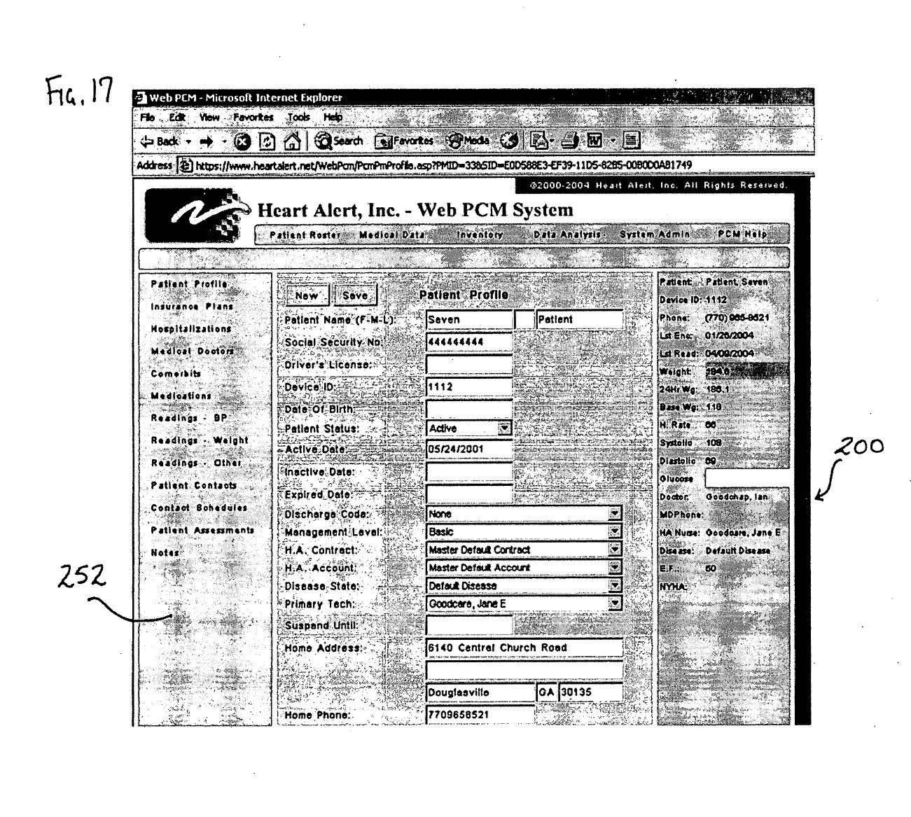

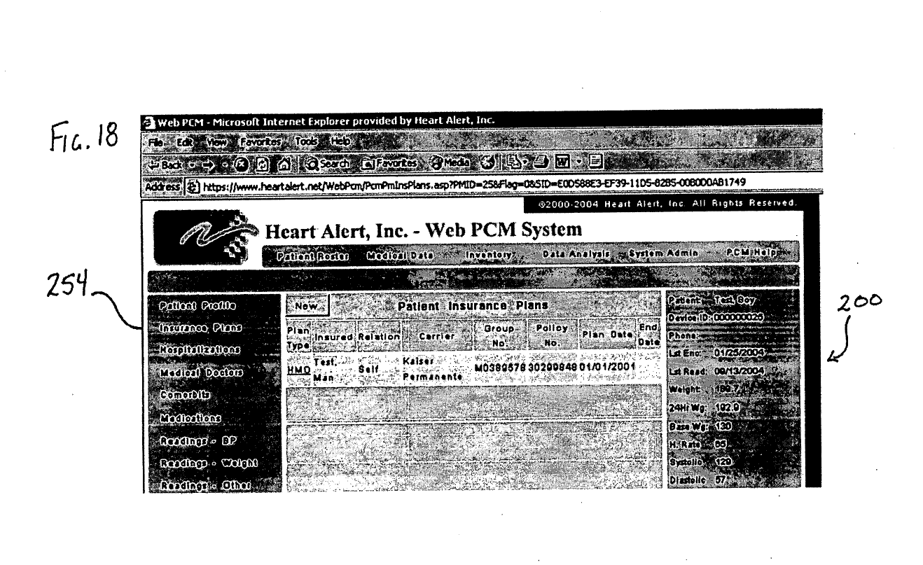

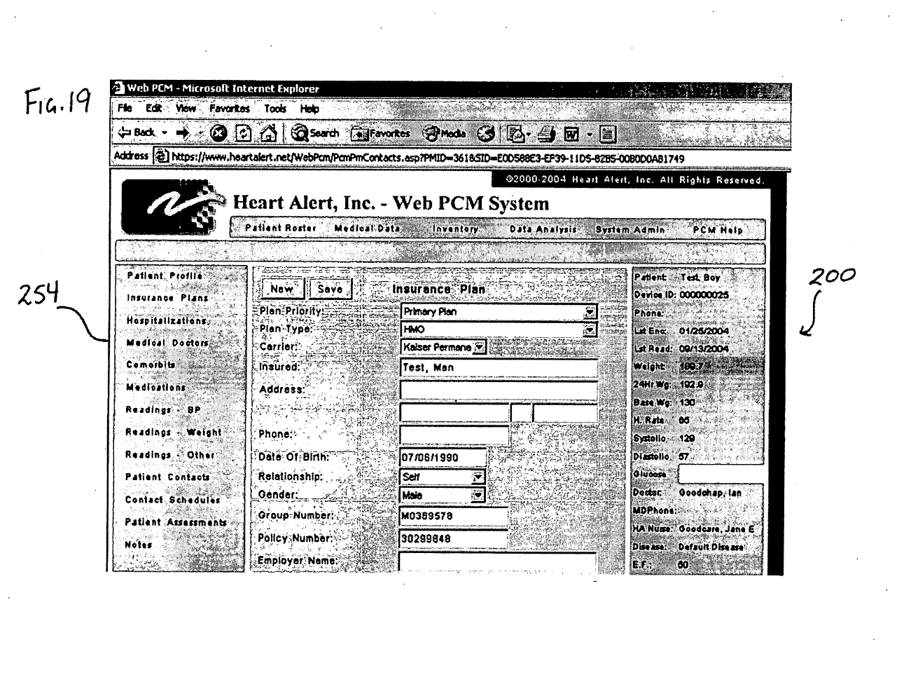

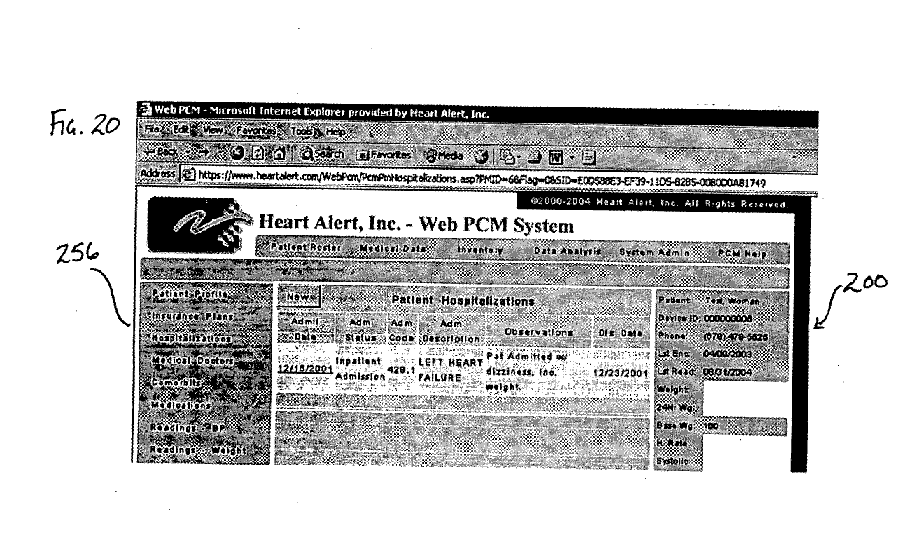

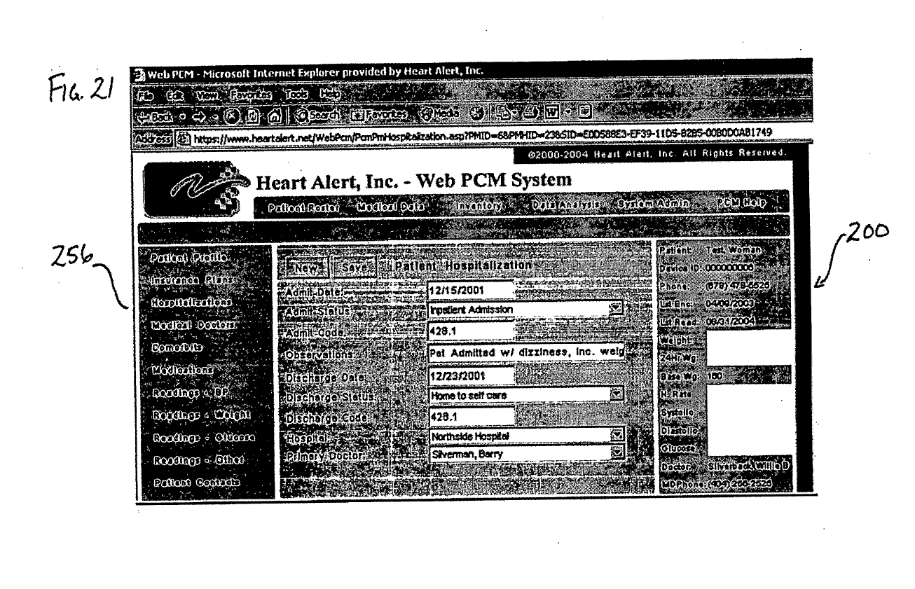

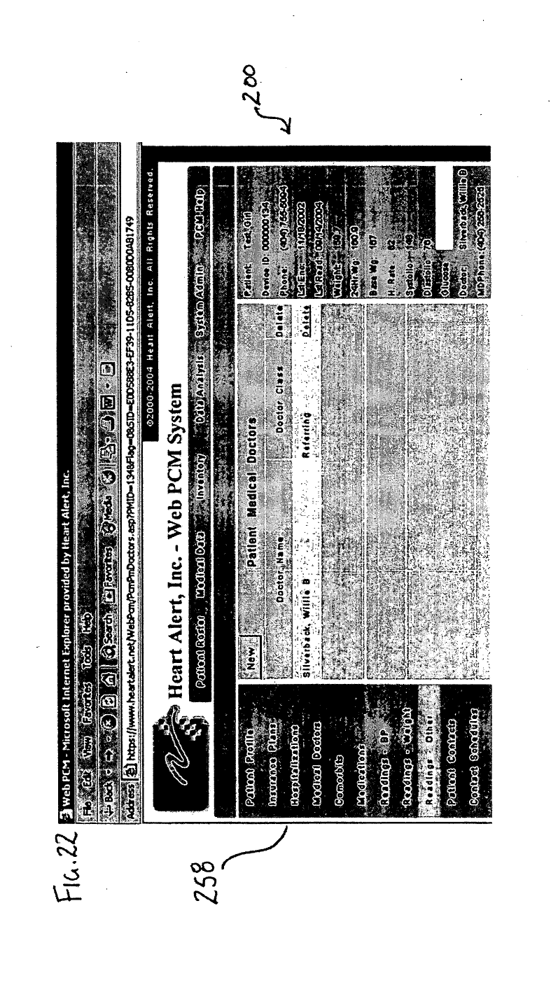

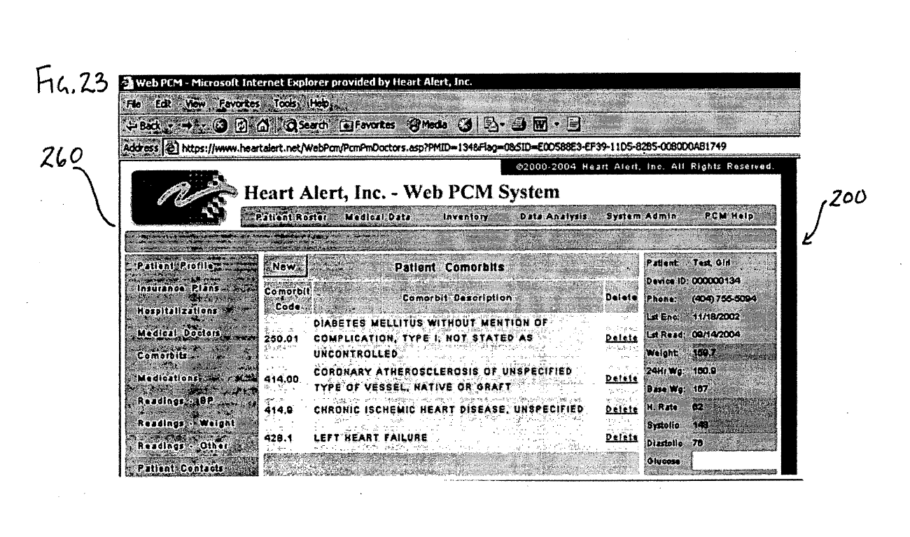

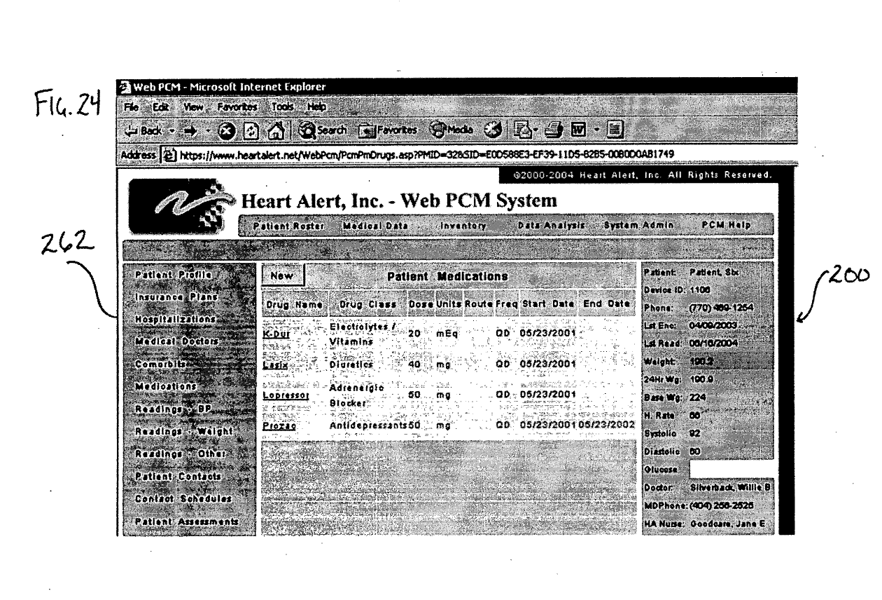

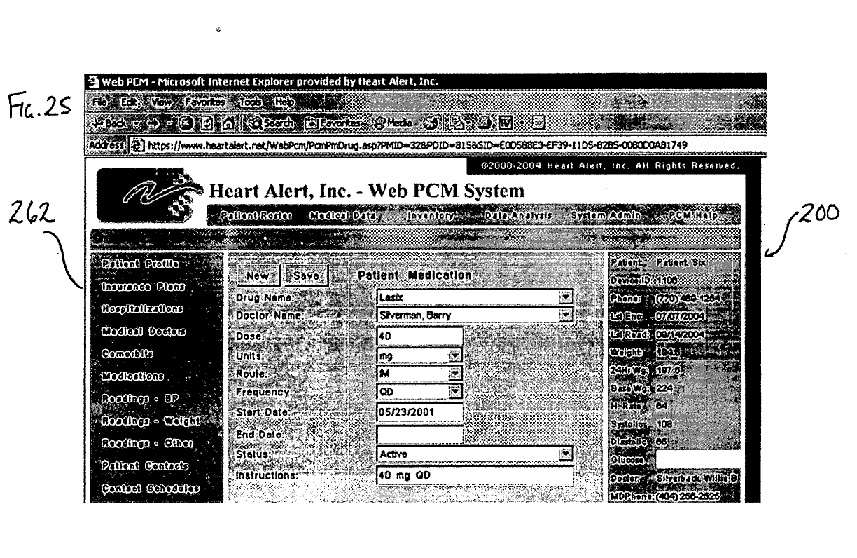

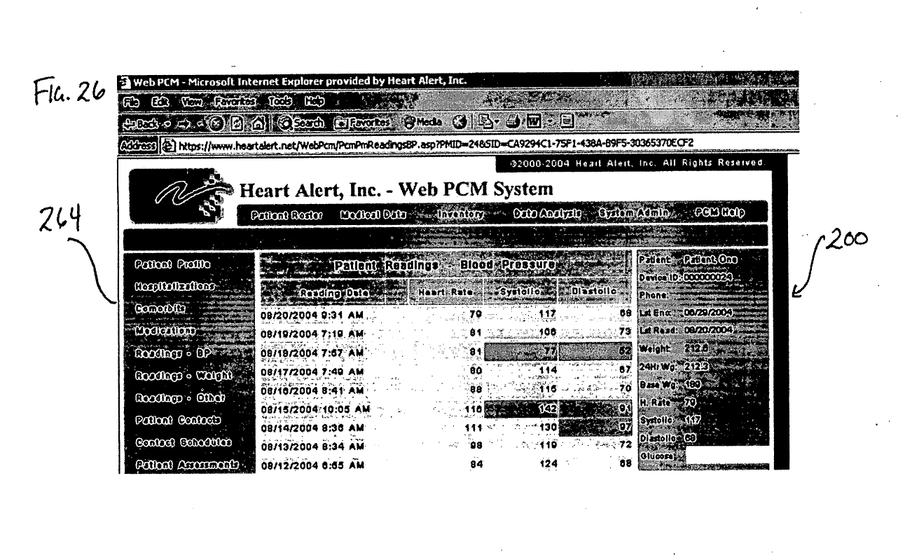

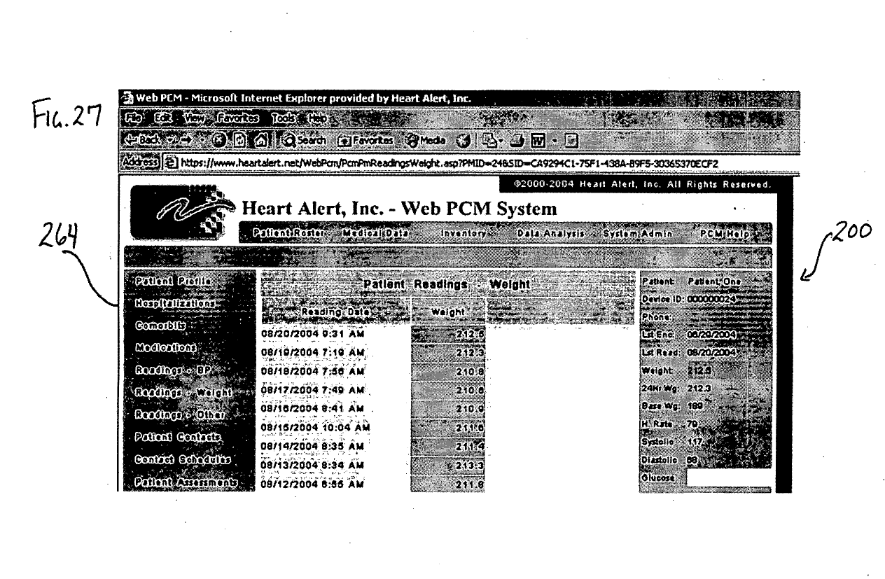

More specifically, patient profile 252 of patient maintenance interfaces 250 enables users to manage demographics information on the patient (see FIG. 17); insurance plans interface 254 enables users to manage insurance coverage data (see FIGS. 18 and 19); hospitalizations interface 256 enables users to manage the patient's hospitalization history (see FIGS. 20 and 21); medical doctor's interface 258 enables users to manage all physicians that have access to an individual patient (see FIG. 22); comorbid conditions interface 260 enables users to manage any comorbid conditions the patient suffers from (see FIG. 23); medications interface 262 enables users to manage all medications taken by the patient (see FIGS. 24 and 25); and, readings interfaces 264 display physiologic readings monitored on the patient, including weight, blood pressure, heart rate, glucose, airflow, ejection fraction, among others, wherein values that fall outside “normal” parameters are shaded in red (under) or blue (over) (see FIGS. 26 and 27).

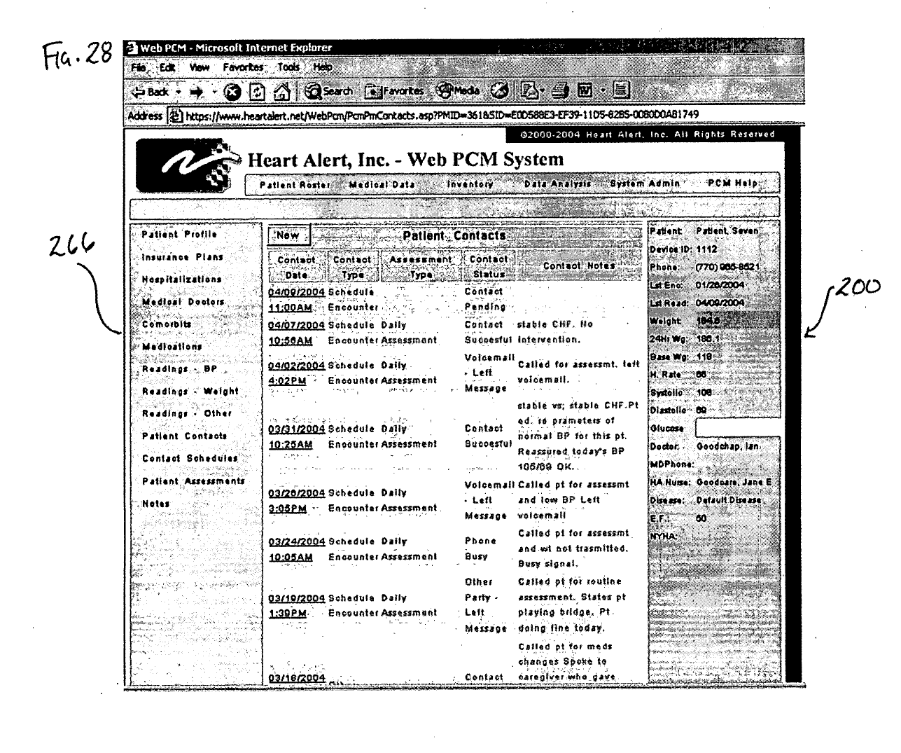

Referring now more specifically to FIG. 28, patient contacts interface 266 of patient maintenance interfaces 250 enables users to manage all communication between the disease management entity, the nurses and the patient. Users can manage contacts, assessments, surveys, follow up on encounters, and the like. Users can further select to view the full version of each encounter and contact in order to obtain a complete picture of each conversation. Further, users can also track any unsuccessful attempts to contact the patient, as well as any upcoming contacts.

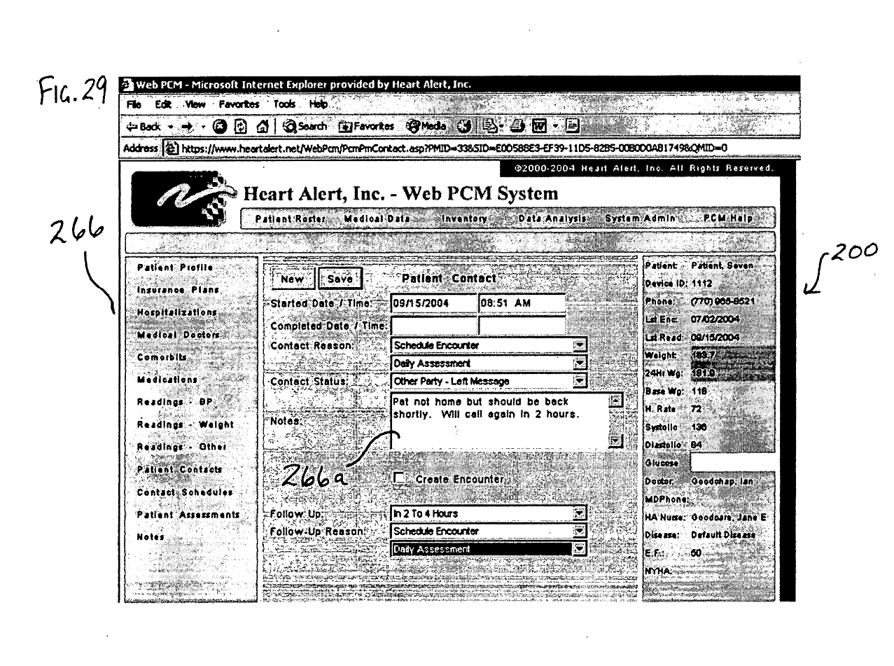

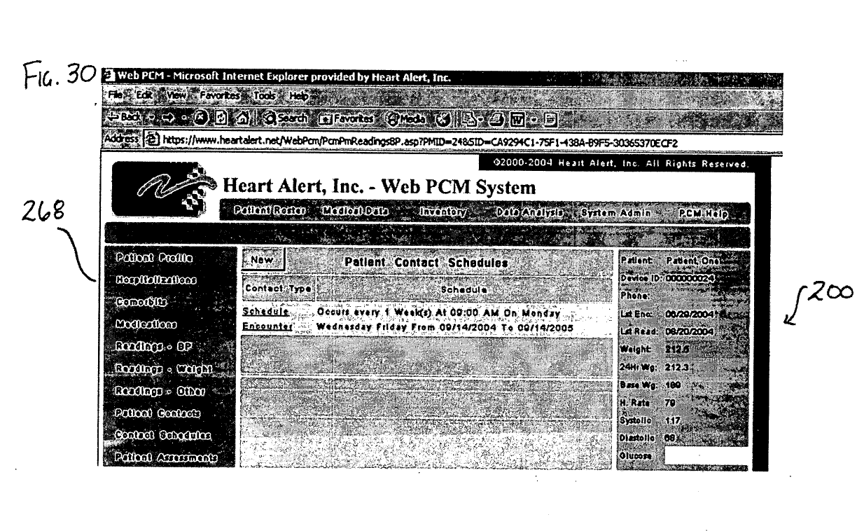

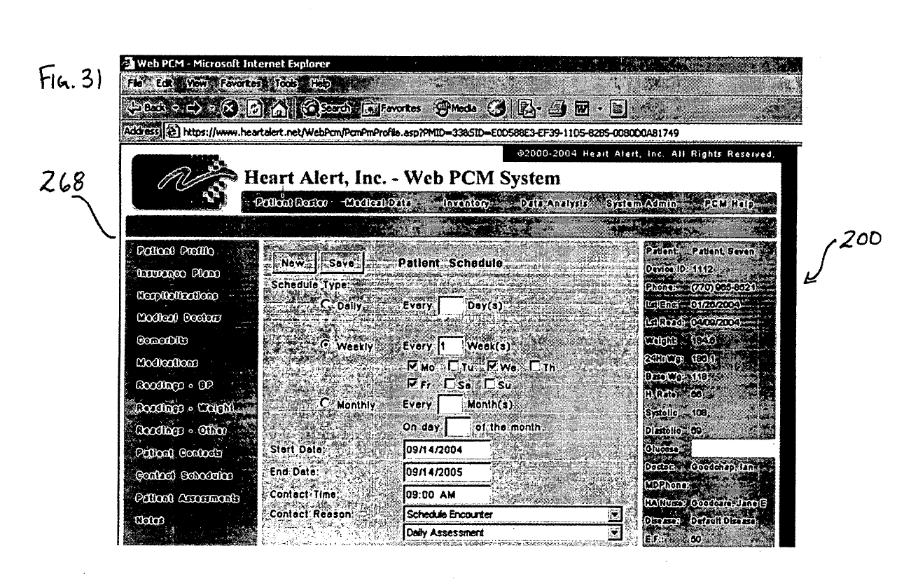

Additionally, patient contact interface 266 provides the patient with options to create any type of scheduled or un-scheduled encounters from a comprehensive list of supported templates. These templates include standing orders, medication protocols, satisfaction surveys, medical history and family history inquiries, chronic condition assessments, co-morbid condition assessments, disease exacerbation and treatment questionnaires, and the like. Moreover, and as best illustrated in FIG. 29, through patient contact interface 266, users may also opt to request physiological reading transmissions from the patient, schedule follow-up contacts, track the patient's admission process, schedule reminders and tasks, or the like, wherein status codes allow users to categorize the results of a contact and a note field 266a allows users to input additional information about the conversation. Further, contact schedules interface 268 of patient maintenance interfaces 250 enables users to schedule re-occurring contacts of any interval and type (see FIGS. 30 and 31).

Referring now more specifically to FIGS. 13A, 32 and 33, with continued reference to FIG. 13, patient assessments interface 270 of patient maintenance interfaces 250 enables users to manage assessments of any type, including standing orders, protocols, scheduled and un-scheduled encounters, and satisfaction surveys. Patient assessments interface 270 preferably supports the following templates: (1) standing orders 270a, which include medical orders received from the referring doctor that detail the level of care, monitoring and management each patient should receive, and wherein standing orders 270a instruct nurses on readings to monitor, physiological thresholds, medication management, dietary and fluid restrictions, and any special directions to be included in the patient care plan; (2) protocols 270b, which include directions on the proper management of each individual medication, and provide nurses with instructions to properly administer Rx titrations, according to specified thresholds and guidelines; and, (3) encounters 270c, which include any scheduled and un-scheduled encounters, and any type of questionnaire, survey, or form utilized during the continuous management of patients.

Referring now to FIG. 13B, with continued reference to FIG. 13, medical data menu or interface 272 of patient maintenance interfaces 250 provides users with tools and interfaces to manage clinical and medical data needed to support daily clinical operations. Generally, medical data menu 272 provides the following interfaces: medical staff interface 272a, utilized to manage information on outside medical users, such as doctors, nurses, and the like; medical facilities interface 272b, utilized to mange information on medical care facilities such as hospitals, clinics, medical groups, and the like; care pods interface 272c, utilized to manage the medical teams (i.e., nurses, technicians and administrative staff) assigned to manage a specific group of patients; medications interface 272d, utilized to manage drugs and medications; diagnoses interface 272e, utilized to manage diagnosis codes; and, questionnaires interface 272f, utilized to manage and create assessment templates used during the daily care of patients.

More specifically, medical staff interface 272a enables users to manage information on outside medical users, such as doctors and nurses. Medical staff interface 272a further enables users to enter new medical staff, assign them to patients, provide them with access to Web-PCM 200, establish electronic notifications whenever one of their patients transmit readings outside normal range, setup delivery of periodic reports, and the like.

Medical facilities interface 272b allows users to manage information on hospitals, clinics, physician groups, and any other type facility that provides medical care to patients. Facilities information is required to support other system processes, such as billing, patient hospitalizations, reporting and outcomes.

Care pods interface 272c enables users to manage the medical teams assigned to manage a specific group of patients, wherein each care pods team preferably includes nurses, technicians and administrative staff responsible for the education, care and overall wellbeing of each individual patient. The care teams are responsible for the daily care and management of the patient, for providing feedback to physicians, and for gathering all relevant clinical and administrative information for the patients assigned to them.

Medications interface 272d enables users to manage information on medications available in the applicable national and/or international market. Accordingly, medications interface 272d provides drug classification information, as well as links to pharmacological databases that contains detailed educational information on each drug, including instructions, use, food and drug interactions, warnings, side effects, and the like.

Questionnaires interface 272f is preferably utilized to create new templates for assessments, standing orders and/or protocols for use during the daily management of patients. Questionnaires can be of any type and length, and can be assigned to a specific group of patients or to the entire patient population.

Referring now still to FIG. 13, inventory menu or interface 274 of patient maintenance interfaces 250 presents users with tools and interfaces to manage telemetric devices and equipment assigned to patients. Inventory menu 274 enables users to search for inventory according to user-defined criteria, add new inventory to the system, and generate inventory related reports, wherein such reports may include: (1) reports by account, which display a list of inventory items assigned to a single account; (2) reports by vendor, which display a list of inventory items from a single vendor; (3) reports by unit number, which searches the inventory database by the unit serial number; (4) reports by unit status, which searches the inventory database by the unit status; (5) inventory alert reports, which display reports listing each type of item and the number of units available in-house, as well as the number of units assigned to patients, to accounts, under repair, lost, or the like; and, (6) new inventory reports, which enable users to enter new inventory items.

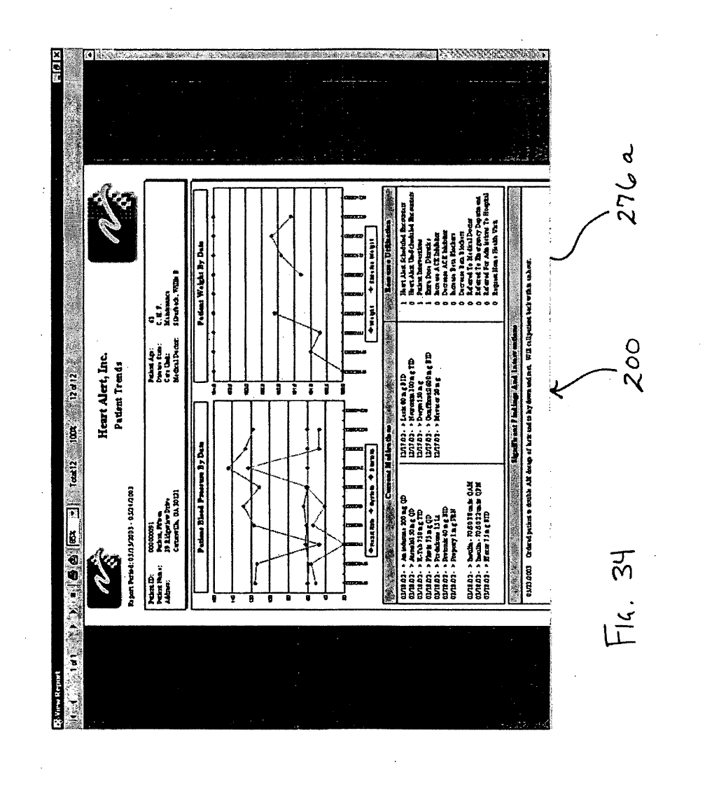

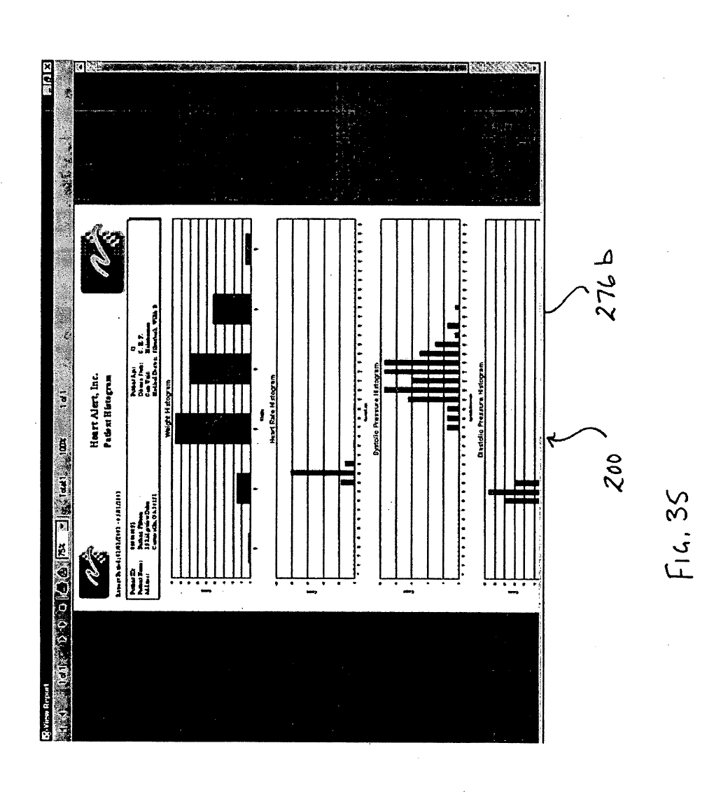

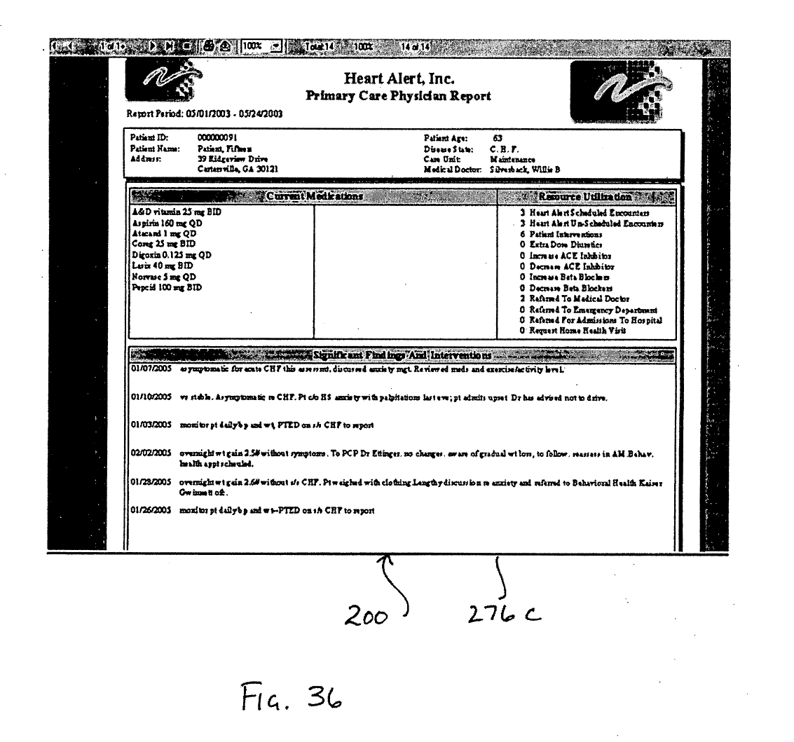

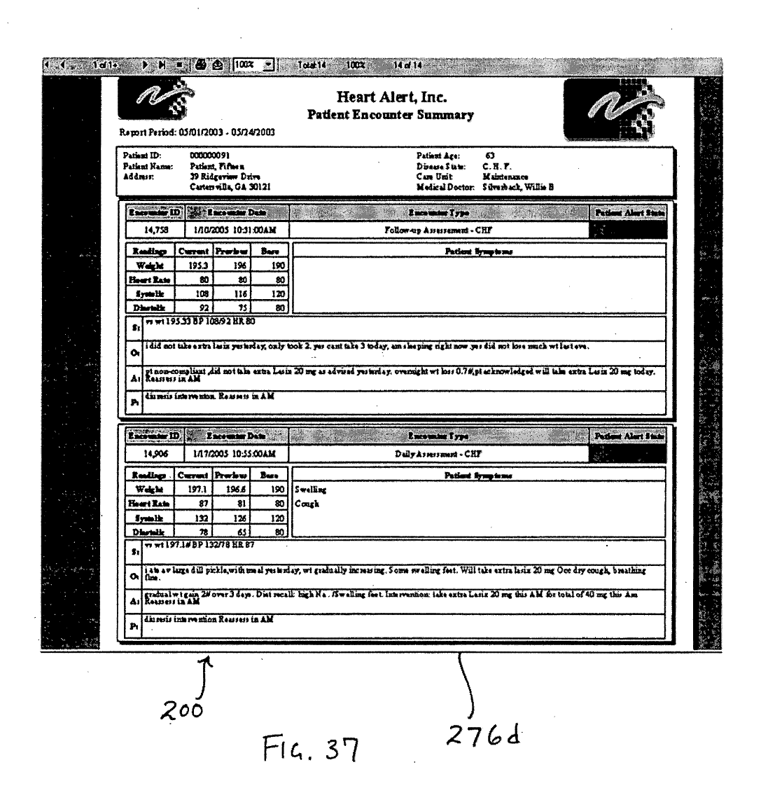

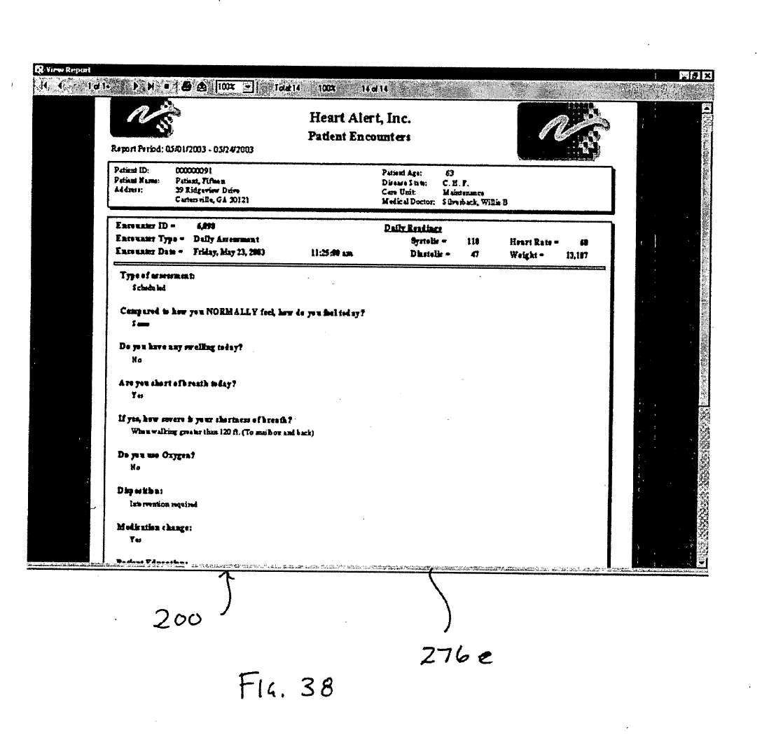

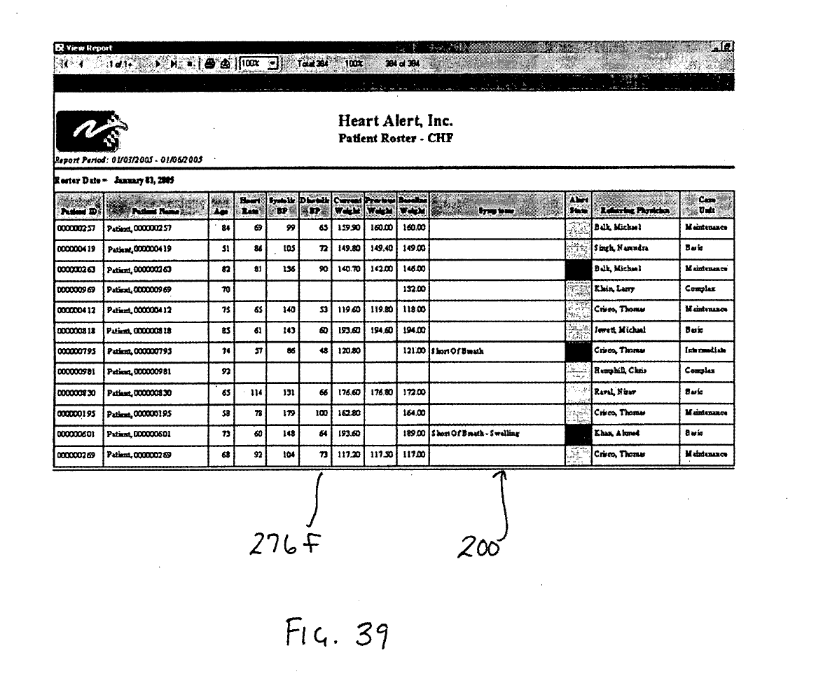

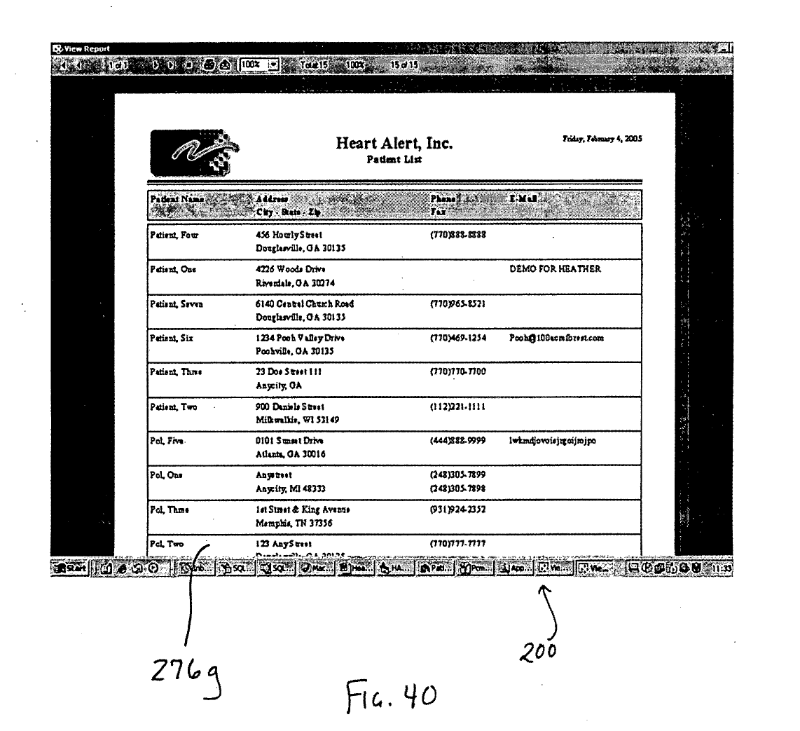

Referring now to FIG. 13C, with continued reference to FIG. 13, data analysis menu or interface 276 of patient maintenance interfaces 250 enables users to select and generate a variety of special reports according to user-defined criteria, wherein such criteria may include managing nurses, doctors, account information and/or contract information. Preferably, the following reports are available through data analysis menu 276: trends reports 276a, which displays patient trends and information on their daily telemetric transmissions, current medication regime, intervention observations, and other information gathered during daily assessments (see FIG. 34); graph reports 276b, which displays graphical representations of the patient's daily telemetric transmissions, for each telemetric device used by the patient (see FIG. 35); primary care physician reports 276c, which display medical overviews of a patient and any observations recorded during the daily management of the patients (see FIG. 36); encounter summary reports 276d, which display a summary or synopsis of each encounter performed on the patients, according to user-defined criteria, such as telemetric readings for the patient at the time of an encounter, condition symptoms, and any observations made by the nurse or other pod member (see FIG. 37); encounters reports 276e, which display a complete transcript of each encounter performed on the patients, according to user-defined criteria (see FIG. 38); patient roster reports 276f, which display daily rosters of patients with their telemetric transmissions, according to user-defined criteria (see FIG. 39); and patient list reports 276g, which provide lists of patients according to user-defined criteria (see FIG. 40). Preferably, reports 276a-276g of data analysis menu 276 may be selectively generated according to patient status (i.e., active or inactive), date range, assigned contract, assigned account, managing nurse, referring physician, and the like.

Referring now to FIG. 13D, with continued reference to FIG. 13, system administration menu or interface 278 of patient maintenance interfaces 250 presents users with tools and interfaces to manage a variety of administrative-related information of system 10. Preferably, system administration menu 278 provides the following interfaces: (1) contracts interface 278a, utilized to manage the disease management entity's contracts and patient billing information; (2) accounts interface 278b, utilized to manage the disease management entity's accounts, including demographics, and patient management defaults (for example, the type of patient care allowed under each account, wherein patients may be assigned to an education-only program, a monitoring program, or a full disease management program); (3) vendor interface 278c, utilized to manage the disease management entity's vendors, including vendor demographics; and, (4) carriers interface 278d, utilized to manage data on insurance carriers and other payers.

Referring now to FIG. 13E, with continued reference to FIG. 13, help menu or interface 280 of patient maintenance interfaces 250 enables users to access documentation on Web-PCM 200 and any functionality therewithin. Preferably, help menu 280 provides the following interfaces: (1) contents interface 280a, utilized to access data contents of the system documentation file; (2) search interface 280b, utilized to search for specific information about Web-PCM 200 or any functionality thereof; and, (3) “about” interface 280c, which provides a summary of system 10 information, including version and copyright disclaimer.

Referring still to FIG. 13, preferably operating in association with clinical information system 100, and software information system C generally, is a voice communications system 300, wherein voice communications system 300 preferably provides the disease management entity with an effective means for managing all inbound and/or outbound calls from physicians, patients, payors, and the like. Specifically, voice communications system 300 preferably permits all patient calls to be delivered to their respective individual pods via suitable telephone and/or broadband communications lines/systems, wherein backup communication lines are preferably provided in cases of emergency and/or failure of the main communication lines.

Voice communications system 300 further preferably utilizes an advanced digital switching system that integrates voice and data communication features. Voice communication features include traditional telephone features, such as transfers and hold and advanced features. Data communication features allow both voice and data to be transmitted over the same system wiring. Preferably, the advanced digital switching system is adapted to accommodate a multitude of inbound and outbound calls, and a plurality of operator stations for handling the calls. Additionally, the advanced digital switching system preferably provides basic calling and answering functions, wherein calls are preferably answered with multi-line telephones which may be standard desk units, cordless telephones, and/or wireless remote. Moreover, calls may be routed into specifically selected pods, wherein each telephone number or other identification number of the patient/caller will be displayed serving to identify the patient to the nurse or pod staff member answering the call.

Voice communications system 300 further preferably provides conferencing call features and call joining features to allow multiple caregivers to participate in the overall patient care process; auto and manual dialing features; paging features; call hold; and, grouping, wherein group calling is utilized to direct incoming patient and physician calls to a specific pod assigned to particular patient responsibilities. Preferably, individual patients and physicians are able to contact each pod by dialing the telephone numbers which will direct the call to the specific station numbers within the pod.

Voice communications system 300 still further preferably provides a call accounting system, wherein the call accounting system is preferably a software application which will be utilized to effectively manage the communications cost associated with the patient treatment method B. Preferably, communications costs are managed by tracking, sorting and compiling usage within the voice communications system not only from a cost of facility standpoint, but also from a Human Resources standpoint. Preferably, a call record processing system collects, stores and produces records of calls and calculates the cost associated therewith utilizing pre-programmed cost factors. Additionally, a report generation system associated with the voice communications system organizes and prints call record information in the following formats: summary report by call activity; detail reports providing detailed call-by-code information for each extension within a pod group; account codes detail report that lists every call associated with each account code; and, facility and cost center reports that provide information about the distribution of calls over a particular pod or the entire cost center.

Clinical information system 100 additionally provides a set of networked communication technologies 400 utilized by the disease management entity to communicate with its constituencies and facilitate patient managements. That is, clinical information system 100 provides a communication system (i.e., telephone communication systems and global networking communication systems, including web site portals, “Ask a Nurse” Chat Capability, and “Ask a Doctor” Chat Capability); satellite communication systems (i.e., virtual physician service communication system, and cellular communication systems); communication network systems (i.e., disease management entity to physician virtual private network, disease management entity to hospital virtual private network, disease management entity to ancillary healthcare provider virtual private network, and disease management entity to payor virtual private network); and, a communication database systems, wherein the disease management entity preferably has access to a health resource data library, pharmaceutical information data library, medical practice data library, and a clinical research organization standards data library.

Preferably, patients requesting to be discharged from the present system and method 10 are provided information on how to manage their disease without the services of the disease management entity utilizing the present system and method 10. Patients are further instructed as to how to contact the disease management entity and/or other healthcare resources in their community for assisting in disease management.

The present system and method 10 further provides an admission process into an “Over the Counter” (OTC) disease management program. Preferably, the OTC program preferably comprises a disease management process designed to meet the needs of the patient on a medical maintenance basis. The services rendered to the patient will preferably be focused on the proactive collection of physiological data, proactive collection of subjective data, the reporting of such data and information to the consumer/patient and his/her physician, the access of the patient to the disease management entity's clinical information management center staff of medical professionals on a twenty-four hour basis, the access to the clinical information system medical library, and a continuing education program designed to meet the consumer's/patient's needs.

It should be recognized that although the present system and method 10 disclosed herein focuses on disease management of heart failure patients, it is contemplated that the present system and method 10 could be modified to manage a variety of chronic diseases, including diabetes and asthma.

Having thus described exemplary embodiments of the present invention, it should be noted by those skilled in the art that the within disclosures are exemplary only, and that various other alternatives, adaptations, and modifications may be made within the scope of the present invention. Accordingly, the present invention is not limited to the specific embodiments illustrated herein, but is limited only by the following claims.

1. A system for patient disease management, comprising:
a software management program for managing patient enrollment procedures and patient treatment procedures;
a network of healthcare professionals;
remote vital sign monitors for receiving and transmitting patient physiological data to said network of healthcare professionals;
standing orders and treatment protocols for systematic implementation of same based upon said patient physiological data transmitted by said remote vital signs monitors to said network of healthcare professionals.

2. The system of claim 1, wherein said software program receives and screens said patient physiological data and compares same against said standing orders and treatment protocols.

3. The system of claim 1, wherein said software program determines whether said patient physiological data is within a parameter prescribed by said standing orders and treatment protocols.

4. The system of claim 3, wherein a non-physician member of said network of healthcare professionals conducts a scheduled encounter with the patient to assess the patient's condition.

5. The system of claim 3, wherein a physician of said network of healthcare professionals conducts a scheduled encounter with the patient to assess the patient's condition and modify said standing orders and treatment protocols, if necessary.

6. The system of claim 1, wherein said software program determines whether said patient physiological data is outside a parameter prescribed by said standing orders and treatment protocols, and provides a warning or alert therefor.

7. The system of claim 6, wherein a non-physician member of said network of healthcare professionals conducts an unscheduled encounter with the patient to determine the cause of said patient physiological data being outside said parameter prescribed by said standing orders and treatment protocols.

8. The system of claim 7, wherein said non-physician member administers patient treatment in accordance with said standing orders and treatment protocols to bring said patient physiological data within said parameter.

9. The system of claim 8, wherein a physician of said network of healthcare professionals conducts an unscheduled encounter with the patient to assess the patient's condition and modify said standing orders and treatment protocols, if necessary.

10. The system of claim 9, wherein a physician of said network of healthcare professionals administers patient treatment.

11. The system of claim 9, wherein a member of said network of healthcare professionals contacts a hospital to coordinate admission of the patient therewith.

12. The system of claim 1, further comprising scheduled and unscheduled encounters with a patient to assess the patient's condition and modify said standing orders and treatment protocols, if necessary.

13. The system of claim 12, wherein said scheduled and said unscheduled encounters comprises a series of assessment questionnaires utilized to assess the patient's condition and modify said standing orders and treatment protocols, if necessary, or implement necessary treatment.

14. The system of claim 1, wherein said system coordinates with a payor to satisfy clinical and financial obligations resulting from implementation of said patient enrollment procedures and said patient treatment procedures.

15. The system of claim 1, wherein said software program, and all data stored, received or otherwise contained therein, is at least partially accessible by an authorized user, said authorized user selected from the group consisting of patients, physicians, payors, said network of healthcare professionals, and combinations thereof.

16. The system of claim 1, wherein said system is implemented via a local network or global networking system.

17. The system of claim 1, wherein said protocols and said standing orders are treatment guidelines customized for each patient by a managing physician, and wherein said protocols are based upon hospital practice, ambulatory clinic practice, physician office practice, consulting with other physicians for co-morbid conditions, or ancillary healthcare providers, and wherein said standing orders are based upon published best practice guidelines.

18. The system of claim 1, wherein said standing orders comprise medical orders received from a referring doctor that detail the level of care, monitoring and management each patient should receive, and wherein said standing orders instruct non-physician members of said network of healthcare professionals on readings to monitor, physiological thresholds, medication management, dietary and fluid restrictions, or any special directions to be included in a patient's care plan.

19. The system of claim 1, wherein said protocols comprise directions on proper management of each patient's medication, and further provide non-physician members of said network of healthcare professionals with instructions on how to properly administer Rx titrations or dosages according to specified thresholds and guidelines.

20. A method for patient disease management, comprising the steps of:
a. transmitting and receiving objective and subjective patient physiological data via remote vital sign monitors; and,
b. comparing said patient physiological data to customized protocols and standing orders particular to a patient.

21. The method of claim 20, further comprising the step of determining whether said patient physiological data is within a parameter prescribed by said customized protocols and standing orders.

22. The method of claim 20, further comprising the step of conducting a scheduled encounter with the patient, wherein a non-physician member of a network of healthcare professionals assesses the patient's condition.

23. The method of claim 20, further comprising the step of conducting a scheduled encounter with the patient, wherein a physician of a network of healthcare professionals assesses the patient's condition and modifies said customized protocols and standing orders, if necessary.

24. The method of claim 20, further comprising the step of determining whether said patient physiological data is outside a parameter prescribed by said customized protocols and standing orders, and providing a warning or alert therefor.

25. The method of claim 24, further comprising the step of conducting an unscheduled encounter with the patient, wherein a non-physician member of a network of healthcare professionals determines the cause of said patient physiological data being outside said parameter prescribed by said customized protocols and standing orders.

26. The method of claim 25, further comprising the step of authorizing said non-physician member to administer patient treatment in accordance with said customized protocols and standing orders to bring said patient physiological data within said parameter.

27. The method of claim 26, further comprising the step of conducting an unscheduled encounter with the patient, wherein a physician of a network of healthcare professionals assesses the patient's condition and modifies said customized protocols and standing orders, if necessary.

28. The method of claim 27, further comprising the step of having said physician administer patient treatment if said step of authorizing said non-physician member to administer patient treatment does not bring said patient physiological data within said parameter.

29. The method of claim 28, further comprising the step of contacting a hospital to coordinate admission of the patient therewith if said step of having said physician administer patient treatment does not bring said patient physiological data within said parameter.

30. The method of claim 20, further comprising the step of coordinating with a payor to satisfy clinical and financial obligations resulting from implementation of said method.

31. A system for patient care and disease management, comprising:
a software information suite comprising a plurality of graphical user interfaces or menus for managing and retrieving data, said data selected from the group consisting of patient data, physician data, payor data, disease management entity data, and combinations thereof.

32. The system of claim 31, wherein said plurality of graphical user interfaces or menus comprises a patient roster interface, wherein said patient roster interface allows users to generate lists of patients according to user specified criteria.

33. The system of claim 31, wherein said plurality of graphical user interfaces or menus comprises a patient profile interface, wherein said patient profile interface enables users to manage demographics information on a patient.

34. The system of claim 31, wherein said plurality of graphical user interfaces or menus comprises an insurance plan interface, wherein said insurance plan interface enables users to manage insurance coverage data for a patient.

35. The system of claim 31, wherein said plurality of graphical user interfaces or menus comprises a hospitalizations interface, wherein said hospitalizations interface enables users to manage a patient's hospitalization history.

36. The system of claim 31, wherein said plurality of graphical user interfaces or menus comprises a medical doctor's interface, wherein said medical doctor's interface enables users to manage all physicians that have access to an individual patient.

37. The system of claim 31, wherein said plurality of graphical user interfaces or menus comprises a comorbid conditions interface, wherein said comorbid conditions interface enables users to manage any comorbid conditions from which a patient suffers.

38. The system of claim 31, wherein said plurality of graphical user interfaces or menus comprises a medications interface, wherein said medications interface enables users to manage all medications taken by a patient.

39. The system of claim 31, wherein said plurality of graphical user interfaces or menus comprises a readings interface, wherein said readings interface displays patient physiological readings monitored and transmitted by remote vital sign monitors to said software information suite.

40. The system of claim 31, wherein said plurality of graphical user interfaces or menus comprises a patient contacts interface, wherein said patient contacts interface enables users to manage all communications between the disease management entity, physician and the patient, and to further manage scheduled contacts, unscheduled contacts, assessments, surveys, encounters, follow-up encounters, and track unsuccessful contacts.

41. The system of claim 31, wherein said plurality of graphical user interfaces or menus comprises a patient assessments interface, wherein said patient assessments interface enables users to manage assessments of any type, including standing orders, protocols, scheduled and un-scheduled encounters, and satisfaction surveys.

42. The system of claim 31, wherein said plurality of graphical user interfaces or menus comprises a medical data menu, wherein said medical data menu enables users to manage clinical and medical data comprising medical staff information, medical facilities information, medication information, diagnosis codes, and questionnaires and assessment templates utilized during daily care of a patient.

43. The system of claim 31, wherein said plurality of graphical user interfaces or menus comprises an inventory menu, wherein said inventory menu enables users to manage all remote vital sign monitors, telemetric devices and equipment assigned to patients.

44. The system of claim 31, wherein said plurality of graphical user interfaces or menus comprises a data analysis menu, wherein said data analysis menu enables users to select and generate reports according to user-defined criteria.

45. The system of claim 44, wherein said reports are selected from the group consisting of trends reports, which display a patient's trends and information based on the patient's daily telemetric transmissions, current medication regime, intervention observations, and other information gathered during daily assessments; graph reports, which display graphical representations of a patient's daily telemetric transmissions for each telemetric device used by the patient; primary care physician reports, which display medical overviews of a patient and any observations recorded during daily management of the patient; encounter summary reports, which display a summary or synopsis of each encounter performed on a patient, according to user-defined criteria; encounters reports, which display a complete transcript of each encounter performed on a patient, according to user-defined criteria; patient roster reports, which display daily rosters of patients with their telemetric transmissions, according to user-defined criteria; patient list reports, which provide lists of patients according to user-defined criteria; and combinations thereof.

46. The system of claim 31, wherein said plurality of graphical user interfaces or menus comprises a system administration menu, wherein said system administration menu enables users to manage the disease management entity's contracts, patient billing information, the disease management entity's accounts, account demographics, patient management defaults, the disease management entity's vendors, vendor demographics, insurance carriers, and other payors.

47. The system of claim 31, further comprising a communication technologies system, said communication technologies system selected from the group consisting of voice communications systems, networked communication technologies, telephone communication systems, global networking communication systems, satellite communication systems, virtual physician service communication systems, cellular communication systems, disease management entity to physician virtual private network, disease management entity to hospital virtual private network, disease management entity to ancillary healthcare provider virtual private network, disease management entity to payor virtual private network, a communication database systems, a health resource data library, pharmaceutical information data library, medical practice data library, a clinical research organization standards data library, and combinations thereof.
